(19) United States
(12) Patent Application Publication
(10) Pub. No.: US 2023/0326542 A1
(43) Pub. Date: Oct. 12, 2023

(54) GENOMIC SEQUENCE DATASET GENERATION

(71) Applicants: Chan Zuckerberg Biohub, Inc., San Francisco, CA (US); The Board of Trustees of the Leland Stanford Junior University, Stanford, CA (US)

(72) Inventors: Daniel Mas Montserrat, Stanford, CA (US); Alexander Ioannidis, Stanford, CA (US); Carlos Bustamante, Berkeley, CA (US)

(21) Appl. No.: 18/042,082

(22) PCT Filed: Sep. 14, 2021
(86) PCT No.: PCT/US21/50182

§ 371 Date: Feb. 17, 2023

Related U.S. Application Data
(60) Provisional application No. 63/078,148, filed on Sep. 14, 2020.

(51) Int. Cl. G16B 5/00; G16B 40/00

(52) U.S. Cl. CPC G16B 5/00; G16B 40/00

(57) ABSTRACT

In one example, a method comprises: receiving an trait indicator; obtaining, based on the trait indicator, a probability distribution of embedding vectors in a latent space, the probability distribution being generated by a distribution generation sub-model of a trained generative machine learning model from an input vector representing a variant segment associated with the trait indicator, the input vector being defined in a variant segment space having a larger number of dimensions than the latent space; obtaining a sample vector by sampling the probability distribution; reconstructing, by a sequence generation sub-model of the trained generative machine learning model and based on the trait indicator, an output vector from the sample vector, the output vector being defined in the variant segment space; and generating a simulated genome sequence based on the output vector.

### CROSS-REFERENCES TO RELATED APPLICATIONS

The present application claims priority from and is a PCT application of U.S. Provisional Application No. 63/078,148, entitled “Genomic Sequence Dataset Generation” filed Sep. 14, 2020, the entire contents of which are herein incorporated by reference for all purposes.

### STATEMENT AS TO RIGHTS TO INVENTIONS MADE UNDER FEDERALLY SPONSORED RESEARCH AND DEVELOPMENT

This invention was made with Government support under grant number HG009080 awarded by the National Institutes of Health. The Government has certain rights in the invention.

### BACKGROUND

Although most sites in a person's deoxyribonucleic acid (DNA) sequence do not vary between individuals, about two percent (5 million positions) do. These are referred to as single nucleotide polymorphisms (SNPs). Human populations all share a common ancient origin in Africa, and a common set of variable sites, but modern human populations exhibit discernible differences in the frequencies of SNP variants at each site in the DNA sequence in their genomes. Because DNA is inherited as an intact sequence with only rare, random swaps in ancestry (between the two parental DNA sequences) at each generation, ancestral SNPs form contiguous segments. As a result, correlations between neighboring sites along the genome, which are typically inherited together, differ between sub-populations around the globe.

Various information can be deduced from the correlations between neighboring sites along the genome. For example, local-ancestry inference uses the pattern of variation observed at various sites along an individual's genome to estimate the ancestral origin of an individual's DNA. In addition, correlations along the genome can influence polygenic risk scores (PRS), genome-wide association studies (GWAS), and many other aspects of precision medicine. Given that the correlations between neighboring genetic variants are ancestry dependent, applying the results of these analyses to an individual's genome may require knowledge of the individual's ancestry at each site along the genome.

Unfortunately, many of the world's sub-populations have not been included in modern genetic research studies, with over 80% of these studies to date including only individuals of European ancestry. This severely restricts the ability to make accurate predictions for the rest of the world's populations. Deconvolving the ancestry of admixed individuals using local-ancestry inference can contribute to filling this gap and to understanding the genetic architecture and associations of non-European ancestries; thus allowing the benefits of medical genetics to accrue to a larger portion of the planet's population.

Various methods for local-ancestry inference exist, such as Hidden Markov Model (HMI) based Analysis of Polymorphisms in Admixed Ancestries (HAPAA), HAPMIX, and SABE, Local Ancestry in adMixed Populations (LAMP) using probability maximization with a sliding window, RFMix using random forests within windows, and Local-ancestry inference Network (LAI-Net) using neural networks. However, these algorithms require accessible training data from each ancestry in order to recognize the respective chromosomal ancestry segments. A major challenge is that many datasets containing human genomic references are protected by privacy restrictions and are proprietary, or are otherwise not accessible to the public. The lack of training datasets can degrade the capability of these algorithms in performing accurate local-ancestry inference.

Accordingly, it is desirable for techniques to generate genome sequence datasets having a more diverse set of genetic variants for different ancestral origins.

### BRIEF SUMMARY

Examples of the present disclosure provide methods, systems, and apparatus for generating simulated genomic sequences having segments of genetic variants (e.g., SNP) for pre-determined trait(s) (e.g., ancestral origin(s)) using a generative machine learning model. The generative machine learning model can receive data representing an input variant (e.g., SNP) segment in a haploid or diploid DNA sequence, as well as information indicating a trait of the segment. The DNA sequence can be obtained from, for example, a genome sequencing operation that provides a genome sequence of the subject, a DNA microarray that contains segments of DNAs, etc. The data representing the input variant segment can include an input vector, with each dimension of the input vector representing a heterozygous site in the genome and being associated with a value indicative of the variant. From the input segment of variants and based on the trait, the generative machine learning model can randomly generate a set of output vectors representing simulated variant segments based on a multi-dimensional probability distribution. The output vectors may have different patterns of variants at the sites in the genome compared with the input variant segment. The simulated variant segments can be variants of the input variant segment and are statistically related to the input variant segment for a particular trait based on the multi-dimensional probability distribution.

According to some examples, certain operations of the generative machine learning model can be performed in a reduced dimensional space (e.g., a latent space), i.e., reduced from number of variants in a segment. For example, an initial mapping can transform N variants to an embedding vector having M dimensions, where M (e.g., 40) is less than N (e.g., 500). For an input variant segment (e.g., having 500 SNPs or other variants), the generative machine learning model can determine a representation of a multi-dimensional probability distribution (e.g., one probability distribution for each dimension of the reduced space), and then, from the one input variant segment, obtain samples of embedding vectors from the multi-dimensional probability distributions. The samples are then reconstructed as the simulated variant segments. In one example, the probability distributions can be modeled as a Gaussian distribution having a multi-dimensional mean and a multi-dimensional variance. In some examples, the probability distribution can have different mean and variance values for each dimension of the reduced space. In some examples, through a training operation based on Kullback-Leibler (KL) divergence, a zero-mean and unit-variance Gaussian distribution (e.g., an isotropic Gaussian distribution) can be achieved. The determination of the particular probability distributions (or one multi-dimensional distribution) can be made based on a mapping of which the parameters are learned in the training operation. Thus, the variant values of the input variant segment can be mapped to a set of distributions (or multi-dimensional distribution). The generative machine learning model can then obtain samples from the multi-dimensional Gaussian distribution, where the samples are reconstructed to generate the output vectors.

In some examples, the generative machine learning model comprises an encoder and a decoder configured as a class-conditional variational autoencoder (CVAE). Both the encoder and the decoder can be implemented as neural network models. The encoder can transform the input vector in a variant segment space to a multi-dimensional probability distribution of embedding vectors in a latent space having a reduced number of dimensions, e.g., by mapping to a mean and width (variance) of the distribution for each of the reduced number of dimensions. For an isotropic distribution, the variance would be the same for each dimension. The distributions in the reduced space can represent variations of the input variant segment. The decoder can obtain samples of embedding vector from the probability distribution, which are then reconstructed to form the output vectors from the samples, the output vectors having the same dimension as the input vector and representing the simulated variant segments.

Both the encoder and the decoder of the CVAE can be trained to fit different patterns of variants to a target multi-dimensional probability distribution, while reducing the information loss in the mapping from variant segment space to the latent space. This can ensure that a simulated variant segment generated by the decoder is statistically related to the input variant segment according to the multi-dimensional probability distribution and can simulate the effect of random variations in the variant segment. The training of the encoder and the decoder can be based on minimizing a loss function that combines a reconstruction error (between the input vector and each of the output vectors) and a penalty for a divergence from a target probability distribution (e.g., based on differences in the parameters (e.g., mean and variance) of the multi-dimensional probability distribution and target values, e.g., of a target probability distribution). The training operation can performed to reduce or minimize the reconstruction error and the penalty of distribution divergence to force the distribution of variant segments generated by the encoder to match (to a certain degree) the target probability distribution, which can be a zero-mean unit-variance Gaussian distribution. The center (mean) and variance of the distribution of the variant segments can be set based on reducing/minimizing the reconstruction error and the penalty of distribution divergence.

To further reduce the distribution error such that the simulated variant segments can follow the target probability distribution more closely, the CVAE can be trained using a class-conditional generative adversarial network (CGAN), which includes the decoder and a discriminator in the aforementioned training operation. The discriminator can also be implemented as a neural network model and can classify whether a variant segment output by the decoder is a real variant segment or a simulated variant segment. The discriminator may be unable to distinguish a real variant segment from a simulated variant segment when the simulated variant segments follow the target probability distribution, at which point the classification error rate of the discriminator may reach a maximum, which means the reconstruction of the decoder is optimal. An adversarial training operation can be performed, in which the parameters of the decoder is adjusted to increase the classification error rate so that the probability distribution in the reduced dimensions approach the target probability distribution, whereas the parameters of the discriminator is adjusted to reduce the classification error rate. The training operation can stop when roughly 50% of the output vectors represent the real variant segment and roughly 50% of the output vectors represent fake/simulated variant segment.

With the disclosed examples, a generative machine learning model can be used to generate a large number of random yet statistically simulated variant segments. For example, through the training operation, parameters of an encoder that maps an input variant sequence to an embedding space for different ancestries, as well as parameters of a decoder that maps an embedding vector to a reconstructed sequence also for different ancestries, can be obtained. The generative machine learning model can receive the a target ancestry as an input. A particular probability distribution (e.g., Gaussian) for that target ancestry can then be selected, and multiple samples of embedding vectors can be obtained from that particular probability distribution. The embedding vectors, as well as the target ancestry, can then be input to the decoder to generate the simulated variant segments. As another example, an input variant segment, as well as its trait, can also be input to the encoder to generate the parameters of a probability distribution, from which the embedding vectors can be sampled, and the sampled embedding vectors as well as the trait can then be input to the decoder to generate the simulated variant segments.

The simulated variant segments can be used for various applications. In one example, the simulated variant segments can be used to train a local ancestry inference machine learning model. As the simulated variant segments can include a diverse set of statistically related variant patterns for different traits, a local ancestry inference machine learning model trained with the simulated variant segments can learn from those variant patterns and predict the trait for a variant segment more accurately.

In another example, the simulated variant segments can also be provided as additional data in genome-wide association studies (GWAS). For example, various statistical techniques can be used to detect underlying relationships among the genomic sequences, traits, and certain target medical/biological traits. To improve the coverage of the training operation, additional variant segments for simulated individuals with (or without) the target medical/biological traits and their traits can be generated using the generative machine learning model, and the additional variant segments can be provided to train the model. The additional variant segments can be used to provide, for example, control data representing variant segments of simulated individuals without the target medical/biological traits and of a target trait, control data representing variant segments of simulated individuals having the target medical/biological traits and but of a different trait, etc.

In addition, the generative machine learning model can provide a portable and publicly accessible mechanism for generating additional variant segments data (for training, for GWAS, etc.). Specifically, data sets containing real human genomic references are proprietary and protected by privacy restrictions. In contrast, the function/model parameters of the generative machine learning model do not carry data that can identify any individual and can be made publicly available. As a result, the generative machine learning model can be made publicly available to generate simulated variant segments to improve training of local-ancestry inference machine learning models, to provide control data for GWAS, etc.

Some examples are directed to systems and computer readable media associated with methods described herein.

A better understanding of the nature and advantages of examples of the present disclosure may be gained with reference to the following detailed description and the accompanying drawings.

### BRIEF DESCRIPTION OF THE DRAWINGS

FIG. 1A and FIG. 1B illustrates examples of single-nucleotide polymorphism (SNP) in a genome and the ancestral origins of the SNPs.

FIG. 2A, FIG. 2B, and FIG. 2C illustrate example analyses of SNP sequences facilitated by examples of the present disclosures.

FIG. 3A, FIG. 3B, FIG. 3C, FIG. 3D, and FIG. 3E illustrate example components of a generative machine learning model to generate simulated SNP sequences according to examples of the present disclosure.

FIG. 4 illustrates an example training operation of the generative machine learning model of FIG. 3A-FIG. 3E according to examples of the present disclosure.

FIG. 5A and FIG. 5B illustrate another example training operation of the generative machine learning model of FIG. 3A-FIG. 3E according to examples of the present disclosure.

FIG. 6 illustrates another generative machine learning model according to some examples.

FIG. 7 shows a sample architecture of a machine learning model that provides relationships among different variants segments according to examples of the present disclosure.

FIG. 8 illustrates an example method of generating simulated SNP sequences, according to some examples.

FIG. 9 illustrates a computer system in which examples of this disclosure can be implemented.

### DETAILED DESCRIPTION

Various information can be deduced from the correlations between neighboring sites along the genome. For example, local-ancestry inference uses the pattern of variation observed at various sites along an individual's genome to estimate the ancestral origin of an individual's DNA. In addition, correlations along the genome influence polygenic risk scores (PRS), genome-wide association studies (GWAS), and many other aspects of precision medicine can be deduced.

For each segment of a genome, a trait can be assigned (e.g., ancestral origin, a biomedical trait, a demographic trait, or other phenotype). Examples are provided for ancestral origin, but techniques described herein also apply to other traits. Synthetic sequences can be generated corresponding to a given trait(s) based on an input sequence, which can be obtained by sequencing cellular DNA or cell-free DNA (e.g., from plasma) of a subject with the trait(s).

The aforementioned local-ancestry inference operations, as well as genome-related medical studies such as computation of PRS and GWAS, can be facilitated with a large genome sequence datasets having a diverse set of genetic variants for different ancestral origins. For example, a local-ancestry inference machine learning model can be trained using a diverse set of statistically related SNP patterns for different ancestral origins, which allows the machine learning model to learn from those SNP patterns and predict the ancestral origin for a SNP segment more accurately. Moreover, the SNP patterns for subjects with known traits can also be used as data for a GWAS study, e.g., to provide data for statistical analyses to detect underlying relationships among the genomic sequences, ancestral origins, and certain biological/medical traits and ancestral origins. However, the availability of data sets containing real human genomic references is typically limited as those data are proprietary and protected by privacy restrictions.

Examples of the present disclosure provide methods, systems, and apparatus for generating simulated genomic sequences having segments of genetic variants (e.g., SNP) for pre-determined ancestral origin(s) using a generative machine learning model. The generative machine learning model can receive data representing an input SNP segment in a haploid or diploid DNA sequence, as well as information indicating an ancestral origin of the segment. The DNA sequence can be obtained from, for example, a genome sequencing operation that provides a genome sequence of the subject, a DNA microarray that contains segments of DNAs, etc. The data representing the input SNP segment can include an input vector, with each dimension of the input vector representing a site in the genome and being associated with a value indicative of the SNP variant. From the input segment of SNPs and based on the ancestral origin, the generative machine learning model can generate one or more output vectors representing simulated SNP segments. The output vectors may have different patterns of SNP variants at the sites in the genome compared with the input SNP segment. The simulated SNP segments can be variants of the input SNP segment that are statistically related to the input SNP segment for a particular ancestral origin.

According to some examples, the generative machine learning model can generate a representation (e.g., mean and variance) of a multi-dimensional probability distribution based on a transformation of the variants of the input SNP segment to a reduced space (embedding/latent space), and then obtain samples of embedding vectors from the probability distribution. Simulated SNP segments are then reconstructed from embedding vector samples (e.g., by a decoder) as the simulated SNP segments. In one example, the multi-dimensional probability distribution can be a Gaussian distribution having a mean and a variance computed determined from a mapping of the input SNP segment, where parameters of the mapping function can be determined based on a training that compares an accuracy of reconstruction. The generative machine learning model can then obtain samples from the Gaussian distribution to generate embedding vectors that are then reconstructed to form the output vectors.

In some examples, the generative machine learning model comprises a first sub-model and a second sub-model, both of which can be implemented as a neural network model. The first sub-model can include an encoder configured to map the input vector a multi-dimensional probability distribution of embedding vectors in a latent space. The latent space can have a reduced number of dimensions relative to the number of SNP sites represented in the input SNP segment. While reducing the number of dimensions, the mapping can still retain information indicative of the pattern of SNP variants of the input vector in the embedding vector. In a case where the probability distribution comprises a Gaussian distribution, the encoder can determine, based on the pattern of SNP variants in the input vector, a mean and a variance of a distribution for each dimension of the embedding vector. Different probability distributions (e.g., different Gaussian distributions having different means and variances for different dimensions of the latent space) can be determined for different SNP sequences. In some examples, the ancestral origin can be input to the encoder with the input vector generate the parameters of a distribution of embedding vectors for that ancestral origin. Multiple probability distributions can be generated by the encoder for different ancestral origins.

In addition, the second sub-model can include a decoder. The decoder can obtain samples of embedding vector from a probability distribution. The probability distribution can be output from the encoder based on encoding an input SNP segment and an ancestral origin, or from a probability distribution previously generated from the encoder based on other input SNP segments and selected based on the ancestral origin and the SNP sites. The decoder can then reconstruct, from the samples of embedding vector, the output vectors having the same dimension as the input vector representing the input SNP segment. As part of the sampling operation, a random function can be implemented based on the parameters to generate random samples of the embedding vectors. The random function can be part of or external to the decoder. As part of the reconstruction operation, the decoder can implement a reconstruction function to map, based on the ancestral origin of the input SNP segment, samples of embedding vectors in the latent space back to the output vectors in the SNP segment space. The output vectors can then represent simulated SNP segments for an ancestral origin.

Both the encoder and the decoder can be trained to maximize the representation of the different patterns of SNP variants in the latent space. In some examples, the encoder and the decoder can be part of a class-conditional variational autoencoder (CVAE), in which different ancestral origins are represented as different classes. The CVAE can be trained using training input vectors representing real SNP sequences for a given ancestral origin in a training operation. The training operation can include a forward propagation operation and a backward propagation operation. As part of the forward propagation operation, the encoder can use the mapping function having an initial set of function parameters to determine the probability distribution of the embedding vectors for the input vectors. The probability distribution can be represented by, for example, a mean and a variance for each dimension of the latent space. The decoder can compute samples of the embedding vectors based on the probability distribution, and use the reconstruction function (having an initial set of function parameters) to compute the output vectors.

The backward propagation of the training operation can adjust the initial function parameters of the mapping function and the reconstruction function to minimize a first loss function. The first loss function can include a reconstruction error component and a distribution error component. The reconstruction error can be generated based on differences between the input vectors and the output vectors, whereas the distribution error can be generated based on a difference between the probability distribution for the embedding vectors and a target probability distribution. In some examples, the distribution error can be computed based on Kullback-Leibler divergence (KL divergence). Through a gradient descent scheme, the function parameters of the encoder and the decoder can be adjusted based on changes in the first loss function with respect to the function parameters, with the objective of minimizing the first loss function. The training can be repeated for training input vectors for different ancestral origins, to determine different function parameters of the mapping function and the reconstruction function for different ancestral origins representing different classes.

The training of the encoder and the decoder based on a combination of the reconstruction error and the distribution error allow the encoder to map an input SNP segment to a probability distribution having target properties (e.g., being isotropic) based on reducing the distribution error, while the probability distribution can be centered based on the embedding vector of the input SNP segment based on reducing the reconstruction error. With such arrangements, the simulated SNP segments (e.g., generated by the CVAE from an input SNP segment given an ancestral origin, or generated by a decoder based on an input probability distribution selected based on an ancestral origin), can include a diverse set of SNP pattern variants, yet the SNP pattern variants remain statistically related based on a target probability distribution.

To further reduce the distribution error such that the simulated SNP segments can follow the target probability distribution more closely, the CVAE can be trained using a class-conditional generative adversarial network (CGAN), which includes the decoder and a discriminator, This training of the CGAN can be performed in the aforementioned training operation, a separate training operation from the encoder, or in a separate loop of training (e.g., where the multiple training iterations occurs for the VAE, then multiple training iterations for the CGAN, back to the VAE, and so on). The discriminator can be a third sub-model of the generative machine learning model and can also be implemented as a neural network model. During the training operation, as part of the forward propagation operation, the decoder can compute random samples of embedding vector and reconstruct output vectors representing the simulated SNP segments. Moreover, the discriminator can determine whether an output vector represents a real SNP segment. The discriminator may be unable to distinguish a real SNP segment from a simulated SNP segment when the simulated SNP segments follow the target probability distribution, at which point the classification error rate approaches 50%.

The target of the training operation at the CGAN is for the output vectors to conform to a target probability distribution (e.g., isotropic Gaussian). To reach the target, an adversarial training operation can be performed in which the parameters of the decoder are adjusted to increase the classification error (based on making the simulated SNP segments more similar to the real SNP segments), while the parameters of the discriminator is adjusted to decrease the classification error. The reconstruction function parameters of the decoder can be adjusted according to a second loss function that decreases when the classification errors at the discriminator increases. Moreover, the model parameters of the discriminator can also be adjusted, in the same training operation, according to a third loss function that decreases when the classification error decreases. The adversarial training operation can be stopped when the classification error rate approaches 50%.

Other variants besides single nucleotide polymorphisms (SNPs) can be used. The variants can be any genetic data at a site, which can correspond to a genomic position or range of positions. Examples of various types of variants include a base, a deletion, an amplification (e.g., of short tandem repeats), an insertion, an inversion, and methylation status. It is possible for a site to include more than one value, e.g., a particular allele of a SNP and particular methylation status. These can be considered different variant values that occur at a same variant site, or the sites can be considered different as they relate to a different type of variant. Either way, the vector of variant values would have the same overall length. Thus, a variant segment can include any set of variant sites (e.g., that are sequential), where the variant sites can have different variant values for one or more types of variants.

### I. Examples of SNP Sequences

A single-nucleotide polymorphism (SNP) may refer to a DNA sequence variation occurring when a single nucleotide adenine (A), thymine (T), cytosine (C), or guanine (G) in the genome differs between members of a species.

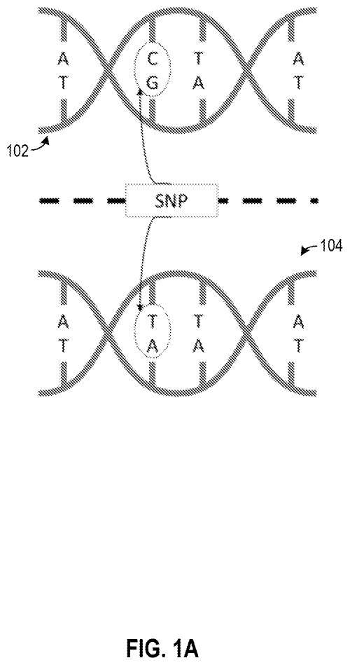

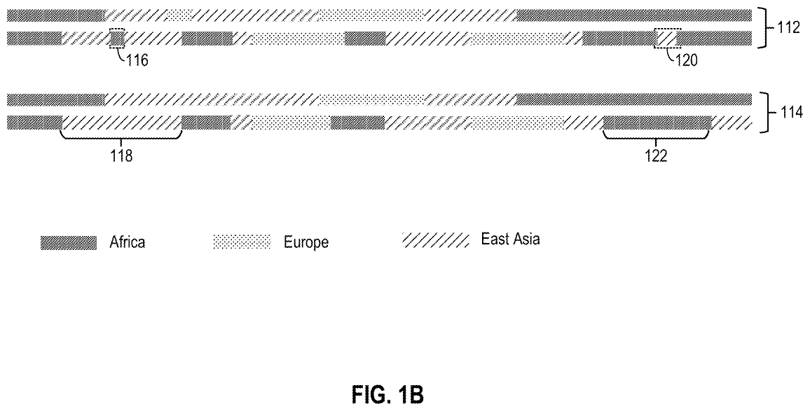

FIG. 1A illustrates an example of SNP. FIG. 1A illustrates two sequenced DNA fragments 102 and 104 from different individuals. Sequenced DNA fragment 102 includes a sequence of base pairs AT-AT-CG-CG-CG-TA-AT, whereas sequenced DNA fragment 104 includes a sequences of base pairs AT-AT-CG-CG-TA-TA-AT. As shown in FIG. 1A, DNA fragments 102 and 104 contain a difference in a single base pair (CG versus TA, typically referred to as C and T) of nucleotides. The difference can be counted as a single SNP. A SNP can be encoded into a value based on whether the SNP is a common variant or a minority variant. The common variant can be more common in the population (e.g., 80%), whereas the minority variants would occur in fewer individuals. In some examples, a common variant can be encoded as a value of −1, whereas a minority variant can be encoded as a value of +1.

Modern human populations, originating from different continents and different subcontinental regions, exhibit discernible differences in the frequencies of SNP variants at each site in the DNA sequence in their genomes, and in the correlations between these variants at different nearby sites, due to genetic drift and differing demographic histories (bottlenecks, expansions and admixture) over the past fifty thousand years. Because DNA is inherited as an intact sequence with only rare, random swaps in ancestry (between the two parental DNA sequences) at each generation, ancestral SNPs form contiguous segments allowing for powerful ancestry inference based on patterns of contiguous SNP variants.

FIG. 1B illustrates an example group of ancestral origins among segments of SNPs of an admixed pair of chromosomes of an individual: one from each parent of the individual. Group 112 illustrates the true ancestral origins of genetic material at different SNP sites of the individual, as may be determined by analyzing a genome of the individual. The individual's genome can be determined by sequencing DNA from the individual's tissue. In the example of FIG. 1B, the ancestral origins of the SNP segments may include Africa, East Asia, and Europe.

Group 112 can be a first stage of classification of the ancestral origins of the SNP segments. As a second stage, a smoothing can be done. Group 114 illustrates the decoded ancestral origins of the SNPs, which can be derived from performing a smoothing operation over group 112 to remove ancestral origin discontinuities in a segment, such as discontinuity 116 (Africa) in segment 118 (East Asia), discontinuity 120 (East Asia) in segment 122 (Africa), etc.

The ability to accurately infer the ancestry along the genome in high-resolution is important to understand the role of genetics and environment for complex traits, such as predisposition to certain illness, certain biomedical traits (e.g., blood pressure, cholesterol level, etc.). This can be due to populations with a common ancestry sharing complex physical and medical traits. For example, certain ethnic groups may have a relatively high mortality of asthma, whereas another ethnic group may have a relatively low mortality of asthma. Elucidating the genetic associations within populations for predisposition to certain illness and biomedical traits can inform the development of treatments, and allow for the building of predictors of disease risk, known as polygenic risk scores. However, because the correlations between neighboring genetic variants (e.g., SNPs) are ancestry dependent, applying these risk scores to an individual's genome requires knowledge of the individual's ancestry at each site along the genome.

The trait can be for any phenotype. For other types of traits, the genome of the subject can still be admixed. For example, segments that have variants associated with cancer (e.g., sequence variants, copy number variants, or structural variants) can be labeled with a trait indicator corresponding to cancer, and other segments can be labeled with a trait indicator of no cancer. For yet other traits, the genome of the subject might not be admixed. For example, a subject with an auto-immune disorder can have all of the segments labeled with a trait indicator for the disorder. A trait can be assigned to a subject in a variety of ways, e.g., based on observation by a doctor, a pathology test, a genomic test, or other type of test.

A subject could have multiple traits, e.g., ancestral origin, a demographic (e.g., height), and biomedical trait (e.g., existence of a condition, such as diabetes). Subjects can be clustered based on the traits that they have. The subject can be labeled with the various traits in any number of ways. For example, one-hot encoding can be used to specify whether each trait exists for a segment. Some traits can be grouped (e.g., whether a condition exists or not, or different age ranges), with only one trait indicator from a group being positive (e.g., 1).

Embodiments can be used to simulate genomic sequences associated with any one or more of these traits without having to use a particular person's genome, thereby preserving privacy. For example, a hospital can have genomic sequences for subjects who have type 2 diabetes, are Native American members of a tribe, and/or have other traits, and the people want to keep their DNA private. Embodiment can create synthetic genomes that have the same properties for these people but are not their personal genomes. These synthetic genomes can be used to train another model that is predictive of the trait in other subjects.

### II. Example Analyses of SNP Sequences

A machine learning model can be used to perform an ancestry-specific analysis of a subject's genome data. Various machine learning models for local-ancestry inference exist, such as Hidden Markov Model (HMM) based Analysis of Polymorphisms in Admixed Ancestries (HAPAA), HAPMIX, and SABE, Local Ancestry in adMixed Populations (LAMP) using probability maximization with a sliding window, and RFMix using random forests within windows.

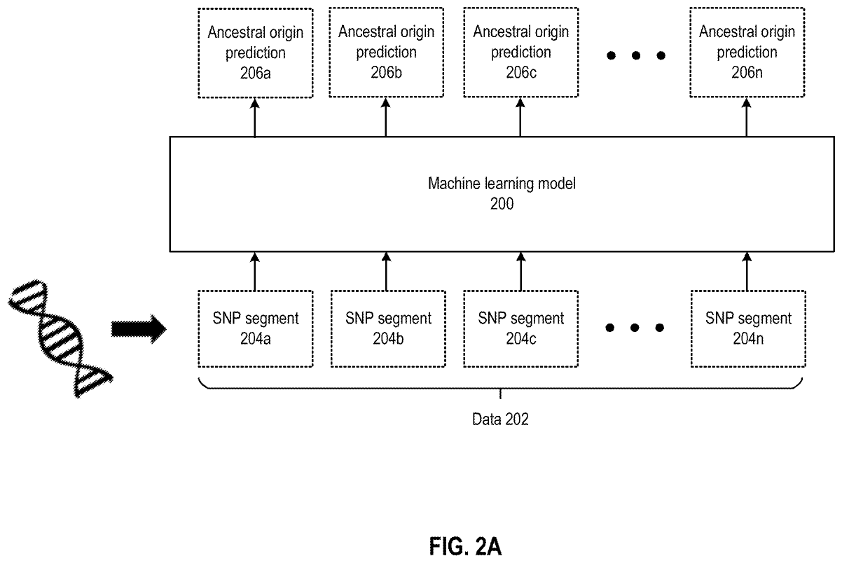

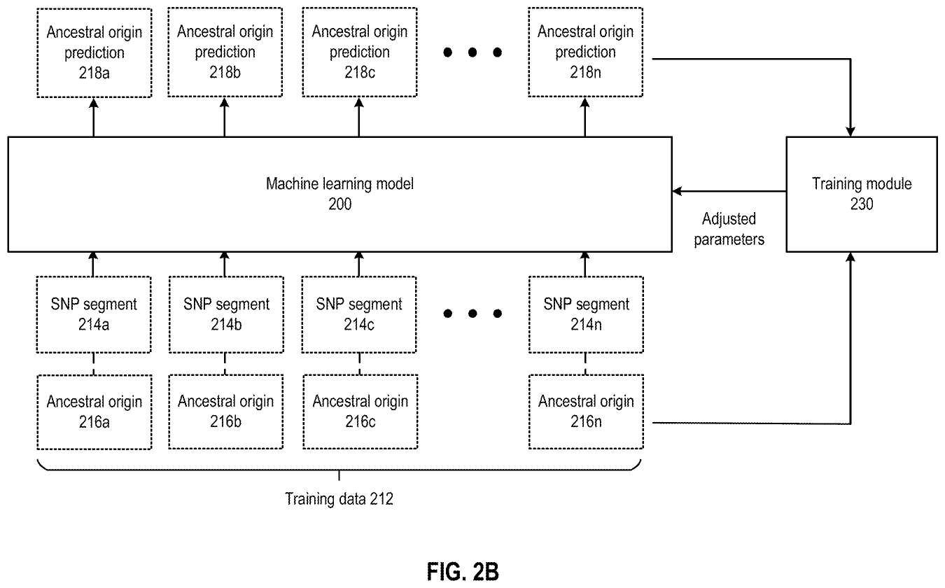

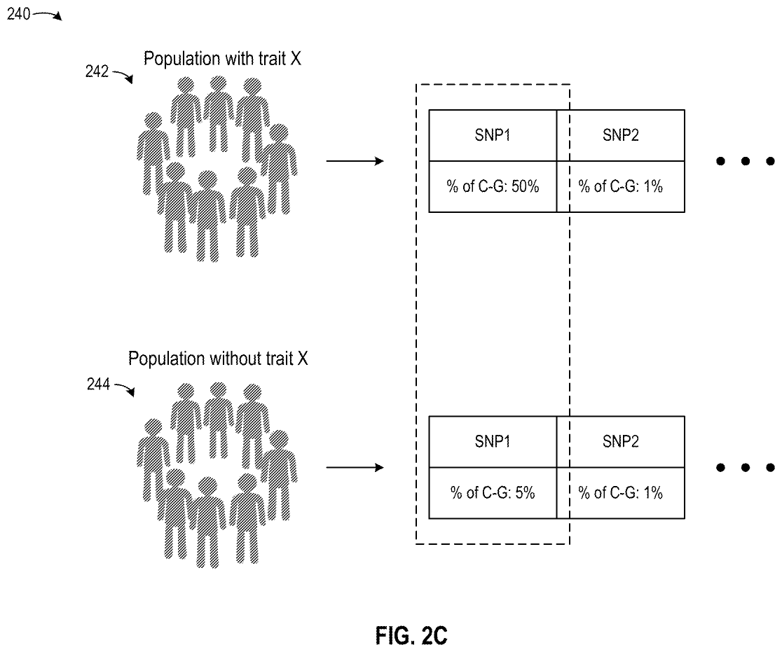

FIG. 2A illustrates a general topology of a machine learning model 200 for performing a local-ancestry inference, according to some examples. As shown in FIG. 2A, machine learning model 200 can receive data 202 representing an input genomic sequence of a subject (e.g., a person). The input genomic sequence may cover a plurality of segments each including a plurality of single nucleotide polymorphism (SNP) sites of the genome of the subject. Each segment may be represented, in data 202, by a sequence of SNP values at the SNP sites, with each SNP value specifying a variant at the SNP site.

Data 202 can include SNP segments 204a, 204b, 204c, 204n, etc. For each segment, machine learning model 200 can generate, based on the pattern of the SNP values in the segment and their associated SNP sites, an ancestral origin prediction (e.g., whether a SNP segment is originated from Africa, Europe, or East Asia) for each SNP segment. In FIG. 2A, machine learning model 200 can generate an ancestral origin prediction 206a for SNP segment 204a, an ancestral original prediction 206b for SNP segment 204b, an ancestral origin prediction 206c for SNP segment 204c, and an ancestral origin prediction 206n for SNP segment 204n. The ancestral origin predictions can be concatenated to provide, for example, groups 112 and/or 114 of FIG. 1B. Each segment can include a same or different amounts of variants (e.g., SNPs). Example number of variants in a segment include 50, 100, 150, 200, 250, 300, 400, 500, 1000, 5000, and 10000 sites.

Machine learning model 200 can be trained using genome data of individuals with known ancestral origins to learn various ancestry-specific patterns of SNPs, and to apply the learning to identify ancestry-specific patterns of SNPs from input genome data in more accurate manner.

FIG. 2B illustrates an example training operation. As shown in FIG. 2B, machine learning model 200 can receive training data 212 that include SNP segments 214a, 214b, 214c, and 214n, as well as the known ancestral origins 216a, 216b, 216c, and 216n of each segment. Machine learning model 200 can apply an initial set of model parameters to generate an ancestral origin prediction 218a for SNP segment 214a, an ancestral origin prediction 218b for SNP segment 214b, ancestral origin prediction 218c for SNP segment 214c, and ancestral origin prediction 218n for SNP segment 214n. A training module 230 can compare the ancestral origin prediction and the known ancestral origin for each SNP segment, and adjust the model parameters based on the comparison result. The adjustment can be based on maximizing the percentage of matching ancestral origin predictions among the SNP segments in training data 204.

Local-ancestry inference can be helpful for genome-wide association studies (GWAS). GWAS is a study of a genome-wide set of genetic variants in different individuals to see if any variant is associated with a trait, such as predisposition to certain illness, certain biomedical traits (e.g., blood pressure, cholesterol level, etc.). Accordingly, such studies can associate specific genetic variations with particular diseases. Knowing a predisposition of a particular ancestral origin for a particular disease can help identify whether certain variations are associated with the particular disease.

FIG. 2C illustrates an example of GWAS 240. In FIG. 2C, a population 242 has a trait X, whereas a population 244, which can be a control group, does not. The genome sequences in both populations are then analyzed, and the SNP (if any) for each site is determined. In FIG. 2C, the SNP being counted is the occurrence of a C-G base pair at a DNA site that typically has a T-A base pair (or just C vs. T if just one strand, e.g., Watson strand, is being used). Among population 242 who has a biological/medical trait X, 50% of the individuals have a C-G base pair at a first DNA site (labelled “SNP1”). In contrast, among population 244 who does not have trait X, only 5% of the individuals have a C-G base pair at the first DNA site. Meanwhile, only 1% of both populations 242 and 244 have a C-G base pair in a second DNA site (labelled SNP2). From the study, it can be determined that individuals with the C-G base pair as SNP1 are overrepresented among population 242, which may suggest a strong linkage between the occurrence of the C-G base pair as SNP1 and trait X. Further, given that the correlations between neighboring genetic variants are typically ancestry dependent, it is also desirable that the SNP patterns included in the study are associated with different ancestral origins, and linkages can also be found between the traits and ancestral origins.

A large set of SNP sequences data having a diverse set of SNP patterns for different ancestral origins can be useful to train machine learning model 200 of FIG. 2A and FIG. 2B, and to provide a basis for GWAS 240 of FIG. 2C. Specifically, to improve the performance of machine learning model 200, the training data can include a diverse set of SNP patterns for each ancestral origin. As the model parameters are adjusted based on maximizing the percentage of matching ancestral origin predictions among the input SNP segments, using a diverse set of SNP patterns to train the model parameters enable machine learning model 200 to detect/distinguish a wider variety of SNP patterns, which can improve the accuracy of the ancestral origin prediction.

Moreover, in GWAS 240, both population 242 (with trait X) and population 244 (without trait X) should include individuals having wide variety of SNP patterns. This is to ensure that both populations are representative of the general population, such that the conclusion from the GWAS (e.g., a strong linkage between the occurrence of the C-G base pair as SNP1 and trait X) is applicable to the general population, not just to populations 242 and 244. Moreover, by including individuals having a wide variety of SNP patterns in both populations 242 and 244, it can be ensured that various less frequent SNP patterns are included and accounted for in the analysis. This can further support the conclusion that the occurrence of the C-G base pair as SNP1, rather than other SNP variants, is predominantly linked to trait X. This can improve the specificity of GWAS 240 for individuals of different ancestral origins. For example, by further subdividing the individuals in populations 242 and 244 according to their ancestral origins, GWAS 240 can indicate, for example, the strong linkage between C-G base pair in SNP1 with trait X is only applicable to a particular group of individuals having a certain ancestral origin but not for other group of individuals having a different ancestral origin. In some examples, statistical analysis can be performed, based on the SNP segments of individuals, the individuals' ancestral origins, and their biological/medical traits, to detect relationships among genomic sequences, ancestral origins, and certain biological/medical traits.

Although it is desirable to use a large SNP sequences dataset having a diverse set of SNP patterns for different ancestral origins to train a local-ancestry inference model and to provide a basis for a GWAS, the availability of such datasets is typically limited. Specifically, datasets of SNP sequences are typically obtained from real DNA sequences, which are collected from human beings and contain human genomic references. Such datasets are typically protected by privacy restrictions and are proprietary, or are otherwise not accessible to the public. The availability of SNP sequences datasets for certain groups of populations, such as under-served or sensitive populations, can be especially limited due to various reasons, such as the lack of enrollment of these populations in GWAS. As a result, there can be lack of SNP segment data to train machine learning model 200 of FIG. 2A, as well as the machine learning model for a GWAS, to improve the accuracy of those models.

### III. Genomic Sequence Generation Using Machine Learning

To provide more and diverse sets of SNP patterns for different ancestral origins, simulated genomic sequences are provided. Such simulated SNP patterns can be generated in a particular manner to create realistic SNP patterns, thereby allowing them to be used as training sets that will provide accurate local ancestry inference machine learning models.

To this end, a generative machine learning model can be used to generate simulated genomic sequences having segments of genetic variants (e.g., SNP) for pre-determined ancestral origin(s). The generative machine learning model can receive data representing an input SNP segment in a haploid or diploid DNA sequence, as well as information indicating an ancestral origin of the segment. From the input segment of SNPs and based on the ancestral origin, the generative machine learning model can randomly generate a set of simulated SNP segments, which can include different patterns of SNP variants, based on a probability distribution. The simulated SNP segments can be variations of the input SNP segment and are statistically related to the input SNP segment for a particular ancestral origin based on the probability distribution. The simulated SNP segments can be used to, for example, train a local ancestry inference machine learning model, provide control data in genome-wide association studies (GWAS).

With the generative machine learning model, a set of simulated SNP segments having random SNP patterns can be generated. Due to the random nature, the simulated SNP segments can include a diverse set of SNP patterns, yet the SNP patterns are statistically related to those of a real SNP pattern from a real DNA sequence, such that the simulated SNP segments can provide realistic variants of SNP patterns. Such simulated SNP segments can be used to improve an local-ancestry inference model (e.g., machine learning model 200) and to provide control data for a GWAS (e.g., GWAS 240). Specifically, with the simulated SNP segments, machine learning model 200 can learn from a wider but realistic range of SNP patterns to make an ancestral origin prediction, which can improve the likelihood of machine learning model 200 generating an accurate prediction for a real SNP pattern from a real DNA sequence. Moreover, the simulated SNP segments can also improve a GWAS. For example, the simulated SNP patterns can be associated to particular traits.

### A. General Topology

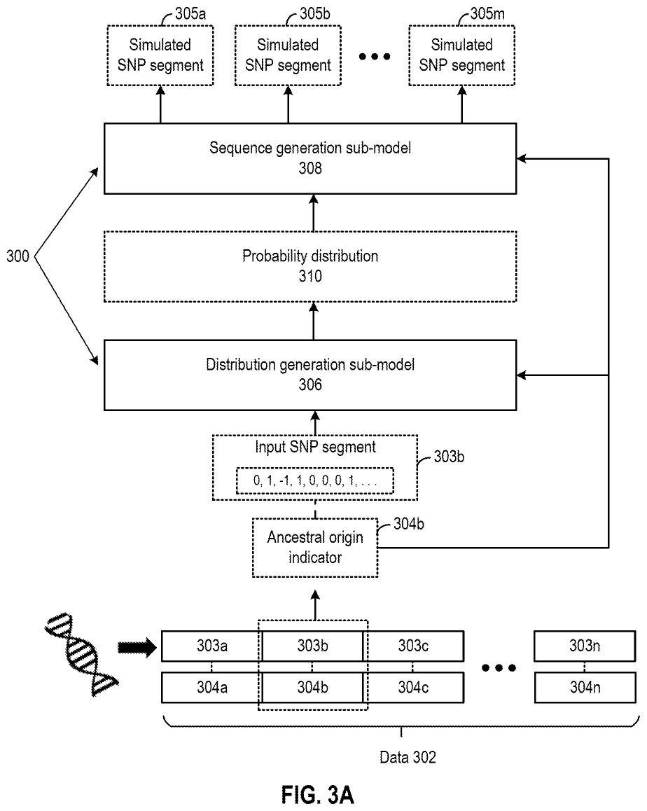

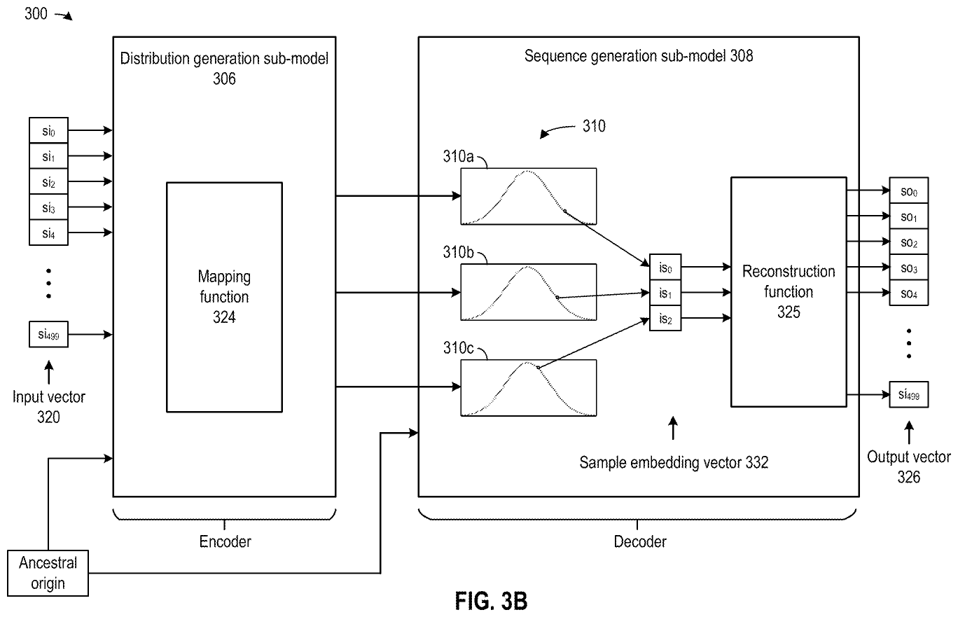

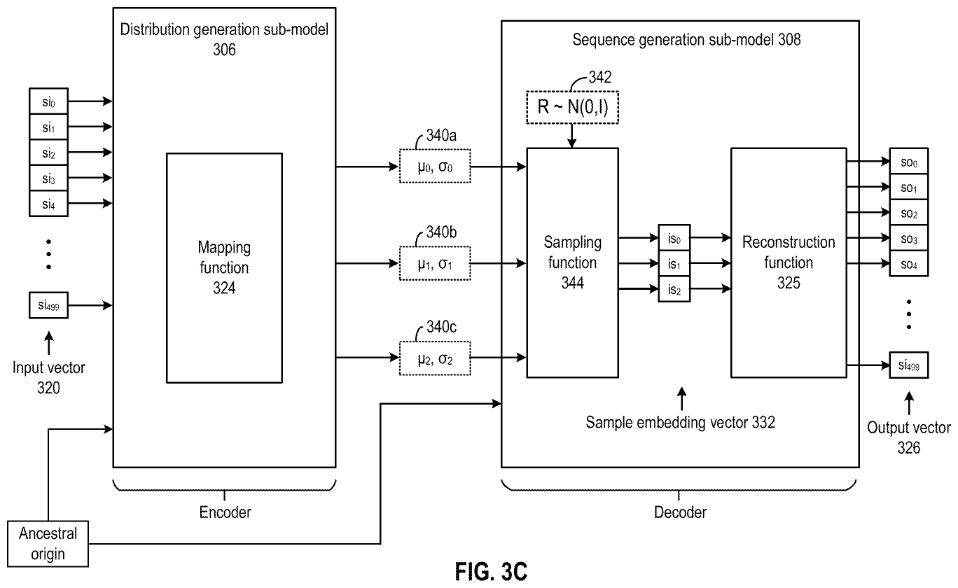

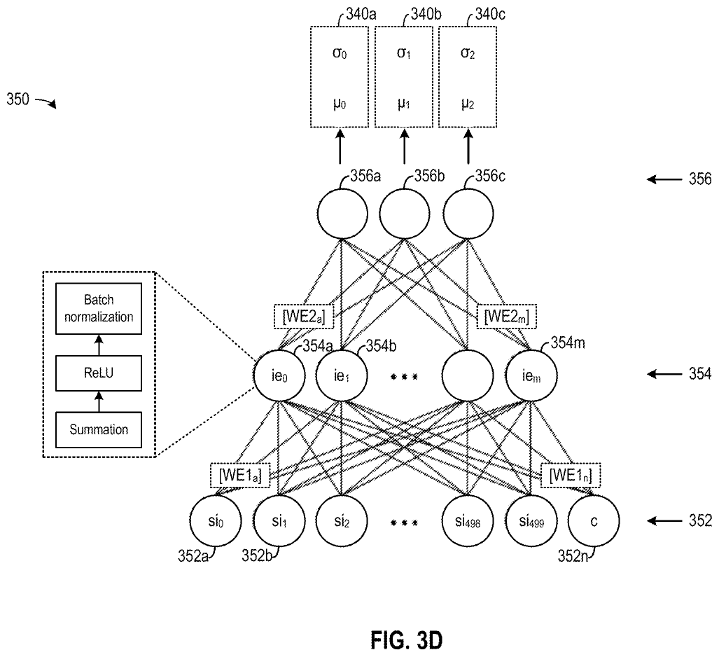

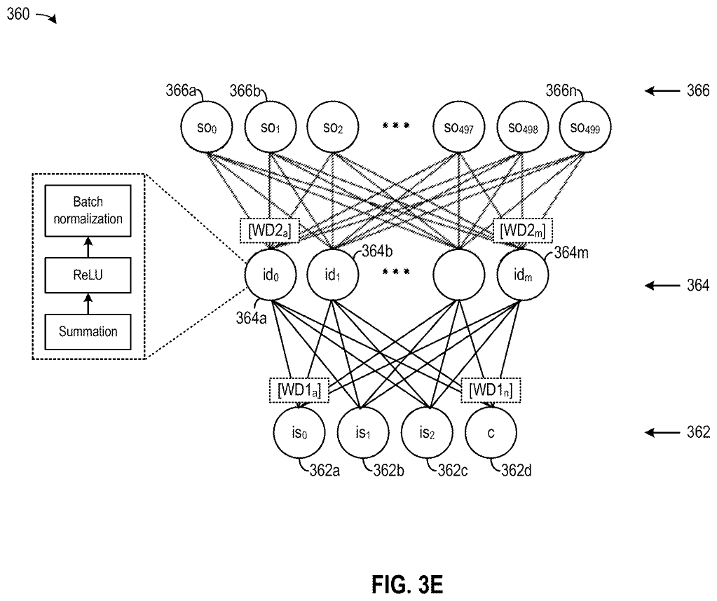

FIG. 3A illustrates a general topology of a generative machine learning model 300 for generating simulated genomic sequences having segments of genetic variants (e.g., SNP) for pre-determined ancestral origin(s). As shown in FIG. 3A, generative machine learning model 300 can receive data 302 representing an input genomic sequence of a subject (e.g., a person) and a group of known ancestral origins for genomic variations within the sequence. The input genomic sequence is divided into a plurality of non-overlapping segments each including a plurality of single nucleotide polymorphism (SNP) sites of the genome of the subject, including input SNP segments 303a, 303b, 303c, 303n, etc. Each segment may be represented, in data 302, by a sequence of SNP values at the SNP sites, with each SNP value specifying a variant at the SNP site (e.g., A, C, T, or G). In addition, each segment is also associated with an ancestral origin indicator which indicates the ancestral origin of the segment. For example, input SNP segment 303a is associated with an ancestral origin indicator 304a, input SNP segment 303b is associated with an ancestral origin indicator 304b, input SNP segment 303c is associated with an ancestral origin indicator 304c, whereas input SNP segment 303n is associated with an ancestral origin indicator 304n.

For each input SNP segment (e.g., input SNP segment 303b) and based on its ancestral origin indicator, generative machine learning model 300 can generate a plurality of simulated SNP segments, including simulated SNP segments 305a, 305b, 305m. Each simulated SNP segment can represent a variation of input SNP segment 303b and is statically related to input SNP segment 303b. Simulated SNP segments for each input SNP segment can be concatenated to form multiple simulated genome sequences, which can correspond to different fictitious individuals.

Data 302 can be obtained from a haploid or a diploid DNA sequence. Data 302 can be obtained from, for example, a genome sequencing operation that provides a genome sequence of the subject, a DNA microarray which contains segments of DNAs, etc. The haplotype information can be encoded to include, for example, a first value representing that a particular SNP is a majority variant (e.g., a value of −1) at a SNP site, a second value representing that the SNP is a minority variant (e.g., a value of +1) at the SNP site, or a third value (e.g., a value of 0) representing that the genomic information is missing at the SNP site. A SNP segment, such as input SNP segment 303b, can include a multi-dimensional vector, with each dimension corresponding to a SNP site and having a value of one of −1, +1, or 0. In addition, ancestral origin indicators 304 can take in various forms. In one example, ancestral origin indicator can include a set of codes indicating an ancestral origin locale out of a set of candidate ancestral origins (e.g., Africa, Europe, East Asia, etc.). In another example, ancestral origin indicator can include geographic coordinates (e.g., longitude and latitude) of the ancestral origin locale. The SNP segments in data 302 can have the same number of SNP values (e.g., 500), or different number of SNP values.

In some examples, generative machine learning model 300 may include two sub-models, including a distribution generation sub-model 306 and a sequence generation sub-model 308. Distribution generation sub-model 306 can accept an input vector representing an input SNP segment (e.g., input SNP segment 303b) and its associated ancestral origin indicator (e.g., ancestral origin indicator 304b). Based on the input vector and the ancestral origin indicator, distribution generation sub-model 306 can determine a multi-dimensional probability distribution 310 in a reduced dimensional space (latent space). Probability distribution 310 can correspond to variations of the input SNP segment. Based on probability distribution 310, sequence generation sub-model 308 can generate a plurality of simulated SNP segments, including simulated SNP segment 305a, 305b, 305m, etc., each representing a random sample of SNPs that is statistically related to the input SNP segment according to probability distribution 310.

Each simulated SNP segment can be regarded as a simulation of random variations in an input SNP segment, in contrast to the input SNP segment, which is extracted from a real DNA sample, e.g., as an input genome sequence of the subject. As to be discussed in details below, distribution generation sub-model 306 can learn the introduction of the random variations to a SNP sequence in a training operation, and determine sub-model parameters that reflect a relationship between a SNP pattern and a probability distribution of variants of the SNP pattern. After the training operation, distribution generation sub-model 306 can apply the sub-model parameters on the SNP pattern in the input SNP sequence to determine parameters of probability distribution 310 of the SNP pattern, whereas sequence generation sub-model 308 can compute random samples of variants of the SNP pattern based on the parameters of probability distribution 310 as simulated SNP sequences.

In some examples, distribution generation sub-model 306 may also receive ancestral origin indicator 304 as well as SNP site information as input without input SNP segment 303, and output probability distribution 310 based on ancestral origin indicator 304. In such examples, distribution generation sub-model 306 may store multiple sets of probability distribution 310 each associated with an ancestral origin indicator and with different SNP sites, and retrieve the probability distribution 310 that corresponds to the input ancestral origin indicator and the input SNP sites. The multiple sets of probability distribution 310 can be previously generated by distribution generation sub-model 306 from other input SNP segments.

### B. Example Components of a Generative Machine Learning Model

In some examples, distribution generation sub-model 306 of generative machine learning model 300 can be configured as an encoder, whereas sequence generation sub-model 308 of generative machine learning model 300 can be configured as a decoder. The encoder and the decoder can combine to operate as a class-conditional variational autoencoder (CVAE).

FIG. 3B illustrates example operations of distribution generation sub-model 306 and sequence generation sub-model 308. Specifically, distribution generation sub-model 306 can implement a mapping function 324 that maps an input vector 320, which represents a SNP segment, to a multi-dimensional probability distribution 310 (represented as 1-dimensional distributions 310a-310c) of embedding vectors in a latent space. The mapping can represent a transformation of the input vector in a SNP segment space having a number of dimensions (defined based on the number of SNP sites represented in the input SNP segment) to embedding vectors in a latent space, which has a reduced number of dimensions.

In some examples (not shown in FIG. 3B), distribution generation sub-model 306 can include multiple mapping functions, each being associated with a class representing an ancestral origin. Distribution generation sub-model 306 can select mapping function 324 to transform the input vector based on the ancestral origin indicator associated with the SNP segment to probability distributions 310 for that ancestral origin. In some examples, distribution generation sub-model 306 can also implement mapping function 324 which receives ancestral origin as part of input vector 320 and generate probability distribution 310 based on both the ancestral origin and the SNP segment represented in input vector 320.

In addition, sequence generation sub-model 308 can implement a reconstruction function 325 to reconstruct an output vector 326 in the SNP segment space from a sample embedding vector 332. Sequence generation sub-model 308 can obtain sample embedding vector 332 from probability distribution 310 output by distribution generation sub-model 306 based on input vector 320, or from another set of probability distributions previously generated by distribution generation sub-model 306 from other input SNP segments. The sampling can be performed by sequence generation sub-model 308, or can be performed by a sampling function separate from sequence generation sub-model 308. The output vectors can represent simulated SNP segments 305a, 305b, 305m, etc., of FIG. 3A to model the effect of random variations of SNP patterns in an input SNP segment.

In the example of FIG. 3B, input vector 320 can include 500 SNP values (si₀, si₁, . . . si₄₉₉) corresponding to 500 dimensions in the SNP segment space, while output vector 326 can include 500 SNP values (so₀, so₁, . . . so₄₉₉) corresponding to the 500 dimensions in the SNP segment space. On the other hand, the latent space can have a reduced number of dimensions (e.g., three dimensions as shown in FIG. 3B). For example, sample embedding vector 332 can include three values (is₀, is₁, and is₂), with each value corresponding to a dimension in the latent space. Same as distribution generation sub-model 306 including multiple mapping functions, sequence generation sub-model 308 can also include multiple reconstruction functions. Sequence generation sub-model 308 can select reconstruction function 325 to reconstruct the output vector from the sample vector 332 based on the ancestral origin indicator associated with the SNP segment. In some examples, sequence generation sub-model 308 can also implement one reconstruction function 325, which receives ancestral origin and sample vector 332 as inputs and generates output vector 326 based on the ancestral origin and sample vector 332.

The transformation and reconstruction operations between the encoder and the decoder, which involves reduction and restoration of dimensions, can create a bottleneck to preserve only the most relevant information in input vector 320 representing the SNP pattern in the embedding vector, and the information can be recovered in the reconstruction of output vector 326. On the other hand, noise information that are not needed to represent the SNP pattern can be discarded during the transformation operation, and is not present in the reconstructed output vector.

Referring back to distribution generation sub-model 306 a probability distribution 310 is multi-dimensional and include a probability distribution for each dimension of the latent space, including probability distributions 310a, 310b, and 310c. In some examples, probability distribution 310 can approach a multi-dimensional isotropic Gaussian distribution, which has the same variance for each dimension, and each dimension can be seen as an independent one-dimension Gaussian distribution centered around a mean value, which can be different among the dimensions of the latent space. An isotropic Gaussian distribution can include a covariance matrix as follows:

Σ=σ²I (Equation 1).

In Equation 1, Σ is the covariance matrix of the isotropic Gaussian distribution, σ² is the common variance among the dimensions, whereas I is an identity matrix. In a case where probability distribution 310 do not match an isotropic Gaussian distribution exactly, each of probability distributions 310a, 310b, and 310c can have a different variance and a different mean.

As to be described below, the parameters of a mapping function 324 can be adjusted to conform probability distribution 310 to a target distribution. Such arrangements can constrain the transformation from the SNP segment space to the latent space to conform with a target probability distribution, to ensure that the latent space is continuous, and that the latent space provide a distribution of different SNP patterns, centered based on, for example, an input SNP segment. Both characteristics allow the decoder to obtain random samples of the embedding vector that provide a realistic SNP sequence, while allowing some variations defined according to probability distribution 310. This allows the random samples to model the effect of random variations of SNP sequences in real DNA samples. Distribution generation sub-model 306 can include multiple distribution generation functions, each being associated with a class representing an ancestral origin. Distribution generation sub-model 306 can select a distribution generation function to generate a probability distribution based on the ancestral origin indicator associated with the SNP segment.

### C. Neural Network Implementation of Generative Machine Learning Model

FIG. 3C, FIG. 3D, and FIG. 3E illustrate additional details of distribution generation sub-model 306 and sequence generation sub-model 308. FIG. 3C illustrates an example of the random sampling operations of embedding vectors between distribution generation sub-model 306 and sequence generation sub-model 308. As shown in FIG. 3C, distribution generation function 330 can generate a representation 340, which includes representations 340a, 340b, and 340c, of probability distribution 310. Representations 340a, 340b, and 340c can include a mean and a variance for, respectively, probability distributions 310a, 310b, and 310c for each dimension of the latent space. For example, representation 340a can include a mean to and a variance σ₀ of probability distribution 310a, representation 340b can include a mean μ₀ and a variance σ₀ of probability distribution 310b, whereas representation 340c can include a mean μ₂ and a variance σ₂ of probability distribution 310c.

In addition, sequence generation sub-model 308 can implement a random function 342 and a sampling function 344 to perform the sampling of probability distribution 310 to generate sample embedding vector 332. In some examples, random function 342 and sampling function 344 can be external to sequence generation sub-model 308. Random function 342 can generate a random matrix R based on an isotropic Gaussian distribution with a zero mean and a unit-variance. Sampling function 344 can generate a sample embedding vector 332, which is a sample of the embedding vector from probability distribution 310, based on multiplying an output random matrix R from random function 342 with a vector of variances from representation 340, and adding the result of the multiplication to a vector of means also from representation 340, based on the reparametrization of CVAE. For example, a sample of sample vector 332 can be generated using sampling function 344 as follows:

$$[ \begin{matrix}{is}_{0} \\ {is}_{1} \\ {is}_{2}\end{matrix} ] = [ \begin{matrix}{μ}_{0} \\ {μ}_{1} \\ {μ}_{2}\end{matrix} ] + [ \begin{matrix}r 0 & 0 & 0 \\ 0 & r 1 & 0 \\ 0 & 0 & r 2\end{matrix} ] \times [ \begin{matrix}{σ}_{0} \\ {σ}_{1} \\ {σ}_{2}\end{matrix} ] \qquad ( Equation 2 )$$

In Equation 2, a value of the first dimension of sample vector 332, is₀, can be computed by adding mean μ₀ with a product of variance σ₀ and a random number r0 of random matrix R. Moreover, a value of the second dimension of sample vector 332, is₁, can be computed by adding mean μ₁ with a product of variance σ₁ and a random number r1 of random matrix R. Further, a value of the third dimension of sample vector 332, is₂, can be computed by adding mean μ₂ with a product of variance σ₂ and a random number r2 of random matrix R. Sequence generation sub-model 308 can generate multiple random matrices R and combine them with the means and variance of representation 340 to generate multiple random samples of embedding vectors, and then reconstruct output vectors based on the sample vectors.

Mapping function 324 and distribution generation function 330 of distribution generation sub-model 306, as well as reconstruction function 325 of sequence generation sub-model 308, can be implemented using a neural network model.

FIG. 3D illustrates an example neural network model 350 of distribution generation sub-model 306 to implement mapping function 324 and distribution generation function 330. Neural network model 350 includes an input layer 352, a hidden layer 354, and an output layer 356. Input layer 352 includes a plurality of nodes such as nodes 352a, 352b, and 352n, which can be a subset of the nodes of the input layer. Hidden layer 354 includes a plurality of nodes such as nodes 354a, 354b, and 354m. Output layer 356 includes a plurality of nodes such as nodes 356a, 356b, and 356c. Each node of output layer 356 can correspond to a dimension of the three dimensions in the latent space of FIG. 3B.

Input layer 352 and hidden layer 354 can implement mapping function 324 to transform an input vector in a SNP segment space to an embedding vector in the latent space. Some of the nodes of input layer 352 can receive an encoded value (e.g., 1, 1, −1) of a SNP value at a particular SNP site of the segment received by the classifier. For example, input node 352a receives an encoded value si₀, input node 352b receives an encoded value si₁, both of input vector 320. In addition, some of the nodes of input layer 352, such as node 352n, receives the ancestral origin indicator associated (labelled c in FIG. 3D) associated with input vector 320.

Each node of input layer 352 is associated with a first set of encoder weights. For example, node 352a is associated with a set of encoder weights [WE1ₐ], node 352n is associated with a set of encoder weights [WE1ₙ]. Each node can scale the input value (SNP value, ancestral origin indicator, etc.) with the associated set of weights to generate a set of scaled values (scaled SNP values), and transmit the scaled values to nodes of hidden layer 354. A larger encoder weight of input layer 352 can indicate that a particular dimension in the SNP segment space include important information about an SNP sequence, and therefore that particular dimension is well represented in the latent space.

Each node of hidden layer 354, which can include one or multiple layers, receives a scaled value from each node of input layer 352, and sum the scaled values to generate an intermediate value (also referred to as an intermediate sum). The intermediate sum can be used to compute probability distribution 310 of the embedding vector at output layer 356. For example, node 354a can compute an intermediate sum, sum₃₅₄ₐ, as follows:

Σ₃₅₄ₐ=Σⱼ₌₀ⁿ(WE1ⱼ×inⱼ) (Equation 3)

In Equation 3, WE can represent a weight value of each set of weights (e.g., [WE1ₐ], [WE1ₙ], etc.) used by each node of input layer 352 to scale an input value inⱼ, which can be either a SNP value (e.g., si₀, si₁, etc.) or ancestral origin indicator c. The combination of ancestral origin indicator with the SNP values in computing intermediate sum can be equivalent to selecting different mapping functions for different ancestral origins.

Each node of hidden layer 354 also implements a non-linear activation function which defines the output of that node given the intermediate sum. The activation function can mimic the decision making of a biological neural network. One example of activation function may include a Rectified Linear Unit (ReLU) function defined according to the following equation:

$$ReLU ( x ) = \{ \begin{matrix}x & for x \geq 0 \\ 0 & for x < 0\end{matrix} \qquad ( Equation 4 )$$

In addition to ReLU, other forms of activation function can also be used included, for example, a softmax function, a softplus function (which can be a smooth approximation of a ReLU function), a hyperbolic tangent function (tanh), an arc tangent function (arctan), a sigmoid function, a Gaussian function, etc. The activation function can be part of mapping function 324 as well to provide a non-linear transformation from the SNP segment space to the latent space, which can improve the filtering of noise information.

In addition to summation and activation function processing, each node of hidden layer 354 can also perform a batch normalization process to normalize the outputs of the hidden layer to, for example, increase the speed, performance, and stability of neural network model 350. The normalization process can include, for example, subtracting a mean of the outputs from each output of the hidden layer node, and dividing by the subtraction results by the standard deviation of the outputs, to generate a normalized output at each hidden layer node. In some examples, the normalization operation can be performed prior to applying the activation function. Based on the activation function processing and batch normalization processing, node 354a generates an intermediate output ie₀, node 354b generates an intermediate output ie₁, whereas node 354m generates an intermediate output ieₘ.

Each node of hidden layer 354 is associated with a second set of encoder weights. For example, node 354a is associated with a set of encoder weights [WE2ₐ], node 354m is associated with a set of encoder weights [WE2ₘ]. Each node can scale the output value of the activation function/batch normalization operation (e.g., ie₀ for node 354a, ie₁ for node 354b, ieₘ for node 354m, etc.) with the associated set of weights to generate a set of scaled values, and transmit the scaled values to nodes of output layer 356.

Each node of output layer 356 can correspond to a dimension in the latent space. Each node of output layer 356 can receive the scaled values from hidden layer 354 and compute a mean and a variance for probability distribution 310 as part of representation 340 of the corresponding dimension of the latent space. For example, node 356a can compute representation 340a, node 356b can compute representation 340b, whereas node 356c can compute representation 340c. Each node can compute the mean and variance based on, for example, summing the scaled output values received from each node of hidden layer 354 based on Equation 3 above.

In some examples, the ancestral origin indicator c is not provided as an input to input layer 352. Instead, distribution generation sub-model 306 can include multiple sets of encoder weights [WE1] and [WE2], each associated with an ancestral origin. The ancestral origin indicator c can be used to select a set of encoder weights for neural network model 350.

FIG. 3E illustrates an example of a neural network model 360 of sequence generation sub-model 308 to implement reconstruction function 325. Neural network model 360 can have a similar architecture as neural network model 350 of FIG. 3D but inverted. Neural network model 360 includes an input layer 362, a hidden layer 364, and an output layer 366. Input layer 362 includes a plurality of nodes including nodes 362a, 362b, 362c, and 362d, which can be a subset of the nodes of the input layer. Each of nodes 362a, 362b, and 362d corresponds to a dimension in the latent space and can receive an element (sample vector value) of a sample vector (e.g., is₀, is₁, and is₂ of sample vector 332) for the corresponding dimension, whereas node 362d receives the ancestral origin indicator c. Hidden layer 364 can include the same number of nodes as hidden layer 354 of neural network model 350 (of distribution generation sub-model 306) and one or multiple layers, whereas output layer 366 includes a plurality of nodes such as nodes 364a, 364b, and 364n. Each node of output layer 366 corresponds to a dimension in the SNP segment space.

Each node of input layer 362 is associated with a first set of decoder weights. For example, node 362a is associated with a set of decoder weights [WD1ₐ], node 362n is associated with a set of decoder weights [WD1ₙ]. Each node can scale the input value (an element of an embedding vector, ancestral origin indicator, etc.) with the associated set of weights to generate a set of scaled values, and transmit the scaled values to nodes of hidden layer 364. The first set of decoder weights can be configured to reverse the second stage of mapping function 324 by hidden layer 354. The combination of ancestral origin indicator with the embedding vector values in computing an intermediate sum (also referred to as an intermediate value) can be equivalent to selecting different reconstruction functions for different ancestral origins.

Each node of hidden layer 364 receives a scaled value from each node of input layer 362, and sum the scaled values based on Equation 3 to generate an intermediate sum. The intermediate sum can then be processed using a non-linear activation function (e.g., ReLU) as well as a batch normalization operation, as in hidden layer 354 of FIG. 3D, to generate an intermediate output. For example, node 364a generates an intermediate output id₀, node 364b generates an intermediate output id₁, whereas node 364m generates an intermediate output idₘ. Each node of hidden layer 364 is also associated with a second set of decoder weights. For example, node 354a is associated with a set of encoder weights [WD2ₐ], node 354m is associated with a set of encoder weights [WD2ₘ]. Each node can scale the output value of the activation function/batch normalization operation (e.g., id₀ for node 364a, id₁ for node 364b, idₘ for node 364m, etc.) with the associated set of weights to generate a set of scaled values (also referred to as scaled sample vector values), and transmit the scaled values to nodes of output layer 366. The second set of decoder weights can be configured to reverse the first stage of mapping function 324 by hidden layer 354.

Each node of output layer 366 then generates a value of the output vector corresponding to one dimension of the SNP segment space based on summing the scaled values from each node of hidden layer 364. For example, node 366a can generate so₀ of output vector 326, whereas node 366b can generate so₁ of output vector 326.

In some examples, the ancestral origin indicator c is not provided as an input to input layer 362. Instead, sequence generation sub-model 308 can include multiple sets of decoder weights [WD1] and [WD2], each associated with an ancestral origin. The ancestral origin indicator c can be used to select a set of decoder weights for neural network model 360.

### D. Training of Class-Conditional Variational Autoencoder

Distribution generation sub-model 306 and sequence generation sub-model 308, configured as a CVAE, can be trained to maximize the representation of the different patterns of SNP variants in the latent space.

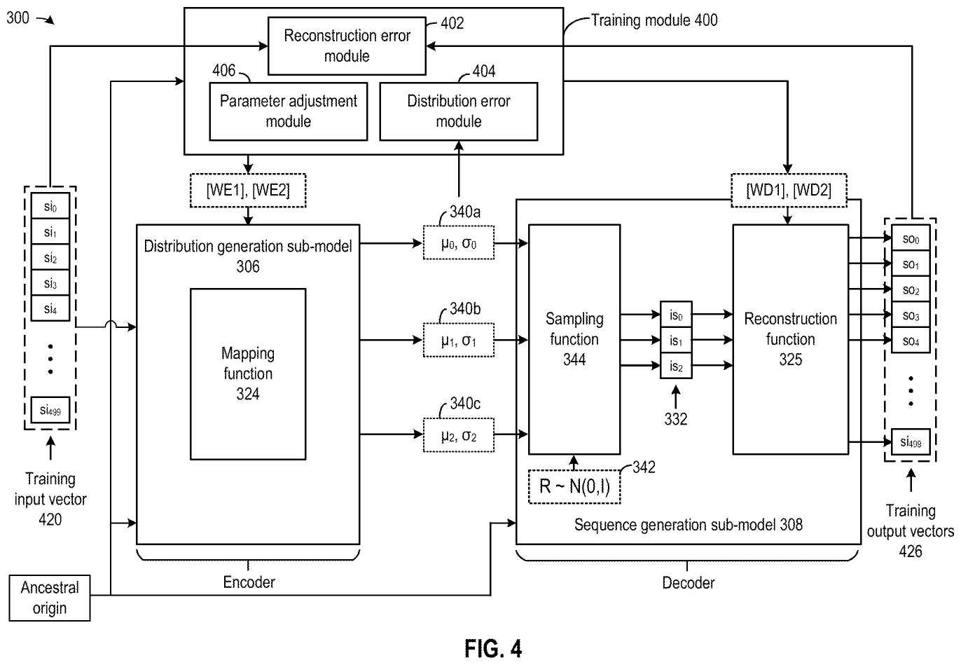

FIG. 4 illustrates an example training operation, in which the encoder and the decoder can be trained by a training module 400 using training input vectors representing real SNP sequences for a given ancestral origin.

The training operation can include a forward propagation operation and a backward propagation operation. As part of the forward propagation operation, distribution generation sub-model 306 can receive a training input vector 420, apply an initial set of parameters of mapping function 324 (e.g., encoder weights [WE1] and [WE2]) on training input vector 420 to generate an initial set of parameters (e.g., mean and variance) of probability distributions 310 of the embedding vectors. Sequence generation sub-model 308 can compute a set of sample embedding vectors 332 based on the probabilistic distribution using sampling function 344, and apply an initial set of parameters of reconstruction function 325 (e.g., decoder weights WD1 and WD2) on the sample embedding vectors to generate a set of training output vectors 426.

The backward propagation of the training operation can adjust the initial function parameters of mapping function 324 and distribution generation function 330 to minimize a first loss function. The first loss function can include a reconstruction error component computed by reconstruction error module 402, as well as a distribution error component computed by distribution error module 404, both of which are part of training module 400. The reconstruction error can be generated by reconstruction error module 402 based on differences, such as means square error, between training input vector 420 and each of training output vectors 426. The distribution error can be generated by distribution error module 404 based on a difference between the probabilistic distribution of the embedding vectors (represented by representations 340) and a target probabilistic distribution. In some examples, the distribution error can be computed based on Kullback-Leibler divergence (KL divergence). One example of the first loss function can be as follows:

[symbol]

_{q}=∥x−\tilde{x}∥²+½Σⱼ^{J}(μⱼ²+σⱼ−log(σⱼ²)−1) (Equation 5)

In Equation 5, [symbol] _{q} represents the first loss function, x can represent an input vector (e.g., training input vector 420), \tilde{x} can represent the output vector constructed from the input vector (e.g., training output vector 426), whereas the first expression ∥x−\tilde{x}∥² can represent the reconstruction error computed by reconstruction error module 402. Moreover, J can represent the last dimension (e.g., J=2 in FIGS. 3A-3C) of the latent space, whereas μⱼ and σⱼ are, respectively, the mean and variance of the jth dimension of the latent space. The second expression ½Σⱼ^{J}(μⱼ²+σⱼ−log(σⱼ)−1) can represent a KL divergence between the Guassian distribution represented by representation 340 and a target isotropic Guassian distribution, computed by distribution error module 404.

In addition, parameter adjustment module 406 can also adjust the initial function parameters of reconstruction function 325 based on minimizing a second loss function. The second loss function can include the reconstruction error represented by the expression ∥x−\tilde{x}∥² output by reconstruction error module 402. As to be described below, the second loss function can also include an adversarial loss component in a case where sequence generation sub-model 308 is also trained using a generative adversarial network (GAN).

Through a gradient descent scheme, a parameter adjustment module 406 can adjust the function parameters of mapping function 324, reconstruction function 325, and distribution generation function 330 (e.g., [WE1], [WE2], [WD1], [WD2], etc.) based on changes in the first loss function and the second loss function with respect to the function parameters, with the objective of minimizing the first loss function and the second loss function. For example, parameter adjustment module 406 can adjust the function parameters in order to achieve a reduction (hence gradient descent) in the first loss function and the second loss function. The training can be repeated for training input vectors for different ancestral origins, to determine different function parameters for different ancestral origins representing different classes.

The training of mapping function 324 and reconstruction function 325, which implements an encoder, based on a combination of the reconstruction error and the distribution error, allows the encoder to map an input SNP segment to the target probabilistic distribution of SNP segment variants, based on reducing the distribution error. Moreover, the probability distribution of SNP patterns can become centered based on embedding vector representing the input SNP segment, based on reducing the reconstruction error in the training operation. With such arrangements, the simulated SNP segments generated by generative machine learning model 300 from an input SNP segment, given an ancestral origin, can include a diverse set of SNP pattern variants defined based on the target probability distribution. Yet the SNP pattern variants remain closely related to the input SNP segment's SNP pattern, as the target probability distribution is centered based on the input SNP segment.

### E. Training Using a Class-Conditional Generative Adversarial Network

To further reduce the distribution error such that the simulated SNP segments can follow the target probabilistic distribution more closely, sequence generation sub-model 308 (e.g., configured as a decoder of a CVAE) can be trained using a class-conditional generative adversarial network (CGAN), which includes the decoder and a discriminator. The discriminator tries to determine a difference between real and simulated SNP segments.

In a CGAN, the decoder and the discriminator can be trained in the same training operation but for opposite objectives. Specifically, the discriminator is to receive a vector representing a SNP segment as input, and classify the input as either a simulated SNP segment generated by sequence generation sub-model 308 (e.g., training output vector 426 of FIG. 4), or a real SNP segment from a real DNA sequence (e.g., training input vector 420). The discriminator can be trained to minimize the rate of classification error (e.g., classifying a real SNP segment as a simulated SNP segment, or vice versa).

When the simulated SNP segments are statistically related to the real SNP segments according to a target probability distribution (e.g., isotropic Gaussian) and the simulated SNP segments have very similar SNP patterns as the real SNP segments (i.e., having low reconstruction errors), it becomes more likely that the discriminator fails to distinguish the simulated SNP segments from the real SNP segments, and the classification error rate increases as result. On the other hand, the decoder can be trained to generate simulated SNP segments to minimize the reconstruction errors and to conform to the target probability distribution, to effectively maximize the classification error rate of the discriminator. Through an iterative adversarial training operation in which the discriminator reduces the classification error, which leads the decoder to restore the classification error by adjusting the decoding weights to make the simulated SNP segments even more, the conformance of the simulated SNP segments to the target probability distribution can be further improved.

The CGAN can be trained in a separate or combined process as the VAE that includes the encoder and decoder. Although the example of a combined process is described below, various training procedures may be used. For example, different input vectors can be used for training the VAE than for training the GAN. And, the distributions that are learned from the training of the VAE might only be randomly sampled when training the GAN.

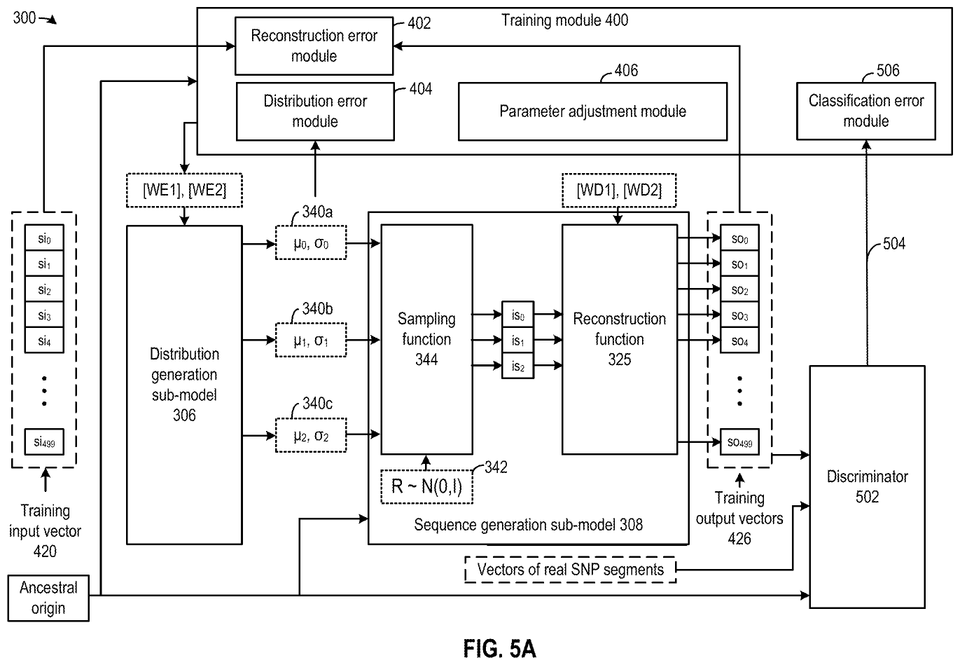

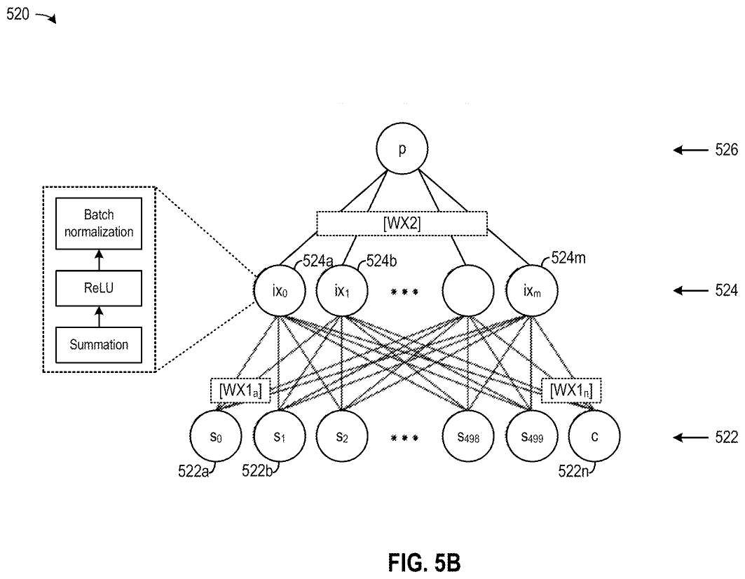

FIG. 5A illustrates additional components to perform the adversarial training operation. As shown in FIG. 5A, a discriminator 502, which can be part of or external to generative machine learning model 300, can form a CGAN with sequence generation sub-model 308, and the CGAN combines with distribution generation sub-model 306 to form a CVAE-CGAN model. During the training operation, as part of the forward propagation operation, distribution generation sub-model 306 can receive a training input vector 420 and generate probability distribution representation 340 in a latent space, whereas reconstruction function 325 can compute samples of output vectors 426 based on probability distribution representation 340 and reconstruction function 325. Discriminator 502 can then perform a classification operation to classify a set of vectors, including vectors representing real SNP segments (e.g., SNP segments extracted from real DNA sequences) such as training input vector 420, as well as training output vectors 426, as whether each vector represents a simulated SNP segment or a real SNP segment, and generate classification outputs 504. The SNP segments can be extracted from a real DNA sequence that is an input genome sequence of the subject.

In some examples, discriminator 502 can be implemented as a neural network. FIG. 5B illustrates an example of a neural network model 520 that can be part of discriminator 502. Neural network model 520 includes an input layer 522, a hidden layer 524, and an output layer 526. Input layer 522 includes a plurality of nodes including nodes 522a, 522b, 522n, etc. Input layer 522 include nodes to receive an input vector representing a SNP segment in the SNP segment space (e.g., node 522a) receives so₀, node 522b receives s₁, etc.), as well as nodes to receive ancestral origin indicator (e.g., node 522n). Hidden layer 524 can provide a non-linear mapping between the input vector and intermediate outputs and can include the same number of nodes as hidden layer 354 of FIG. 3D and hidden layer 364 of FIG. 3E. Output layer 526 includes a single node to compute the probability of the input vector representing a real SNP segment based on the intermediate outputs from hidden layer 524. The probability can be included in classification output 504 to indicate that the input vector represents a real SNP segment if the probability exceeds a threshold, and that the input vector represents a simulated segment if the probability is below the threshold.

Each node of input layer 522 is associated with a first set of discriminator weights. For example, node 522a is associated with a set of discriminator weights [WX1ₐ], node 362n is associated with a set of discriminator weights [WX1ₙ]. Each node can scale the input value (input vector value, ancestral origin indicator, etc.) with the associated set of weights to generate a set of scaled values, and transmit the scaled values to nodes of hidden layer 364. The weights can represent, for example, the contribution of each SNP site in a SNP segment to the classification decision of whether a SNP segment is real or is simulated. The combination of ancestral origin indicator c with the input vector allows discriminator 502 to perform the classification operation based on different criteria for different ancestral origins.

Each node of hidden layer 524 receives a scaled value from each node of input layer 522, and sum the scaled values based on Equation 3 to generate an intermediate sum. The intermediate sum can then be processed using a non-linear activation function (e.g., ReLU) as well as a batch normalization operation, as in hidden layer 354 of FIG. 3D and hidden layer 364 of FIG. 3E, to generate an intermediate output. For example, node 524a generates an intermediate output ix₀, node 524b generates an intermediate output ix₁, whereas node 524m generates an intermediate output idₘ. Hidden layer 524 is also associated with a second set of discriminator weights [WX2], with each node being associated with a weight in the weight set. The weight associated with a node of hidden layer 524 can indicate the contribution of the node to the probability output. Each node can scale the output value of the activation function/batch normalization operation (e.g., ix₀ for node 524a, ix₁ for node 524b, ixₘ for node 524m, etc.) with the associated weight to generate a scaled value, and transmit the scaled value to the single node of output layer 526, which can then generate the probability output (p) by summing the scaled values.

In some examples, the ancestral origin indicator c is not provided as an input to input layer 522. Instead, discriminator 502 can include multiple sets of discriminator weights [WX1] and [WX2], each associated with an ancestral origin. The ancestral origin indicator c can be used to select a set of discriminator weights for neural network model 520.

Referring back to FIG. 5A, training module 400 can include a classification error module 506. During the backward propagation operation, classification error module 506 can determine whether the classification outputs 504 contain errors. Classification error module 506 can determine that a classification output 504 is an error when, for example, the probability indicated in classification output 504 exceeds the threshold (which indicates that a vector is a real SNP segment) but the vector is generated by sequence generation sub-model 308, or when the probability is below the threshold (which indicates that a vector is a simulated SNP segment) when the vector is a training input vector and includes a real SNP segment. The model parameters of discriminator 502 can be adjusted to minimize classification error in classification outputs 504, whereas the function parameters of reconstruction function 325 (e.g., decoder weights [WD1], [WD2], etc.) can be adjusted to maximize classification errors in classification outputs 504.

Specifically, parameter adjustment module 406 can adjust the initial function parameters of reconstruction function 325 ([WD1], [WD2], etc.) based on minimizing the second loss function, which includes the reconstruction error component ∥x−\tilde{x}∥² and an adversarial loss component, as follows:

[symbol]

ₚ=∥x−\tilde{x}∥²+λ₁ log(1−D(z)) (Equation 6)

In Equation 6, [symbol] ₚ represents the second loss function, ∥x−\tilde{x}∥² represents the reconstruction error, z represents a training output vector 426 output by sequence generation sub-model 308, whereas D(z) represents the probability of training output vector 426 representing a real SNP segment, as indicated in classification output 504. The expression (1−D(z)) represents an adversarial loss, which reduces when the classification error increases. For example, in a case where discriminator 502 makes an incorrect classification for training output vector 426 (z), the output probability in D(z) is higher than the threshold, and the expression (1−D(z)) reduces. On the other hand, for a correct classification for training output vector 426, the expression (1−D(z)) increases. λ₁ is parameter that can be set to 0.1 in some examples. Through a gradient descent scheme, parameter adjustment module 406 can adjust the decoder weights (e.g., [WD1], [WD2], etc.). For example, parameter adjustment module 406 can adjust the function parameters in order to achieve a reduction (hence gradient descent) in the second loss function, to reduce the reconstruction error while increasing the classification error.

In addition, parameter adjustment module 406 can also adjust the initial model parameters of discriminator 502 based on minimizing a third loss function, which can be in the form of a binary cross-entropy loss function, as follows:

[symbol]

_{D}=−log(D(x))−log(1−D(z)) (Equation 7)

In Equation 7, [symbol] _{D} represents the third loss function, the expression D(x) represents the probability of a training input vector 420 representing a real SNP segment, as indicated in classification output 504, whereas the expression (1−D(z)) represents the adversarial loss, as in Equation 6. Parameter adjustment module 406 can adjust the initial model parameters of discriminator 502 based on a gradient descent scheme by reducing [symbol] D, which can be achieved by increasing the value of D(x) and/or increasing the value of (1−D(z)). The increase of (1−D(z)) is opposite to the decrease of (1−D(z)) in the second loss function, which leads to the adversarial training operation.

The training operation in FIG. 5A can be performed in multiple phases to minimize the first loss function of Equation 5 (for distribution generation sub-model 306), the second loss function of Equation 6 (for reconstruction function 325), and the third loss function of Equation 7 (for discriminator 502). Specifically, in a first phase, a full forward proprogation operation can be performed on training input vector 420 using an initial set of function/model parameters (e.g., encoder weights [WE1] and [WE2], decoder weights [WD1] and [WD2], and discriminator weights [WX1] and [WX2]). Training output vectors 426, as well as classification outputs 504 for the training output vectors 426 and training input vector 420, can be generated. A full backward propagation can then be performed, in which reconstruction error, distribution error, as well as classification error are determined by training module 400 and propagated back to adjust the parameters for discriminator 502, reconstruction function 325, distribution generation function 330, and mapping function 324. A first set of adjusted function/model parameters can be determined based on minimizing the first loss function (the reconstruction error and KL divergence) for distribution generation sub-model 306.

A second phase of the training operation can then begin, which comprises an adversarial training operation between reconstruction function 325 and discriminator 502. During the adversarial training operation, the decoder weights [WD1] and [WD2], as well as discriminator weights [WX1] and [WX2], can be adjusted (from the first set of adjusted parameters) to minimize both the second loss function for reconstruction function 325 and the third loss function for discriminator 502, but which leds to the conflicting goals for the classification error. The adversarial training operation can be performed in multiple iterations each including a reduced forward propagation operation to compute new training output vectors (e.g., corresponding to output vector 326) as well as classification outputs 504 for the new samples using the adjusted parameters, and a reduced backward propagation operation to adjust only the parameters of reconstruction function 325 and discriminator 502. The adversarial training operation can stop when, for example, roughly 50% of the classification outputs 504 is correct, which leads to a roughly 50% error rate. This can indicate that the training output vectors 426 are so close to training input vector 420 that discriminator 502 cannot distinguish the vectors, and the classification operations become close to a random coin-flip operation, which leads to the 50% error rate.

Although training input vector 420 is shown as being used to train discriminator 502, other real SNP segments can be used for this purpose. Further, a given output vector 426 can be used with multiple real SNP segments to determine the classification error. And, multiple output vectors can be generated using random sampling, and they can be used to determined classification errors against a set of real SNP segments.

When the 50% error rate is achieved at discriminator 502, the second phase of the training operation can stop, and a second set of adjusted parameters for reconstruction function 325 can be obtained. The first phase of the training operation can then restart to propagate the adjustment in the decoder weights of reconstruction function 325 back to distribution generation sub-model 306, to reduce the reconstruction error and the distribution error. The training operation can be repeated for different training input vectors associated with different ancestral origins to, for example, obtain a relationship between an ancestral origin indicator and the probability distribution outputs, the reconstruction outputs, and the classification outputs, to obtain different function/model parameters for different ancestral origins, etc.

In FIG. 4 and FIGS. 5A-5B, generative machine learning model 300 can be trained, using training input vector 420 that includes haploid sequences, to generate haploid sequences. To generate simulated diploid chromosomes, generative machine learning model 300 can be trained separately using each of a pair of haploid sequences of a training diploid sequence to generate variants for each one of the pair of haploid sequences. The variant hapolid sequences can then be paired to generate simulated diploid chromosomes.

In addition, in some examples the simulated SNP sequences generated by generative machine learning model 300 can be post-processed to further improve the diversity of the SNP patterns in the sequences. For example, to generate simulated SNP sequences to represent a number of different individuals, generative machine learning model 300 can be operated to generate simulated SNP sequences for N times of the number of individuals. Pair-wise correlations of the generated SNP sequences can be determined, and 1/N of the set of simulated SNP sequences having the lowest average correlation can be selected as the output. In some examples, N can be set to 2.

### IV. Experimental Results

### A. Experimental Generative Machine Learning Model

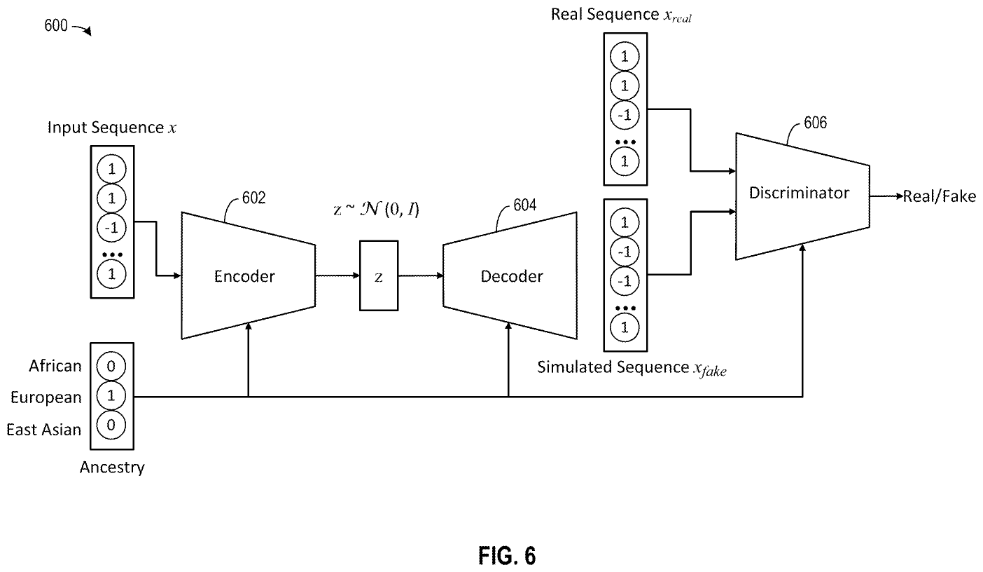

An experimental generative machine learning model 600, illustrated in FIG. 6, is developed and trained. Generative machine learning model 600 can include an encoder 602, a decoder 604, and a discriminator 606. Encoder 602, decoder 604, and discriminator 606 can correspond to, respectively, distribution generation sub-model 306, sequence generation sub-model 308, and discriminator 502 of FIG. 5A and FIG. 5B. Generative machine learning model 600 is trained based on the training operation of FIG. 5A-FIG. 5B (trained as a CVAE-CGAN). In FIG. 6, “z” represent sample vectors 332 obtained from sampling of probability distribution 310 by sampling function 344, as described in FIG. 3C. Generative machine learning model 600 is trained using two different datasets for two different experiments. In each experiment, generative machine learning model 600 generates a set of simulated SNP sequences. A local-ancestry inference model, such as RFMix, is trained with both the simulated SNP sequences and real SNP sequences (SNP sequences extracted from real DNA sequences), and the performance of the performance of the local-ancestry inference model is evaluated to examine the quality of the simulated SNP sequences with respect to the real SNP sequences.

### B. Out-of-Africa Simulation Dataset

In a first experiment, a simulated dataset based on an out-of-Africa simulation is generated and used to train generative machine learning model 600 and a RFMix local-ancestry inference model. The out-of-Africa simulation models the origin and spread of humans as a single ancestral population that grew instantaneously into the continent of Africa. This population stayed with a constant size to the present day. At some point in the past, a small group of individuals migrated out of Africa and later split in two directions: some founding the present day European populations, and another founding the present day East Asian populations. Both populations grew exponentially after their separation.

Following the above out-of-Africa model, three groups of 100 simulated diploid sequences, each representing an individual of single-ancestry, are generated, one group each of African, European and East Asian ancestry, and 300 simulated individuals are generated. The 300 simulated diploid sequences are divided into training, validation and testing sets with 240, 30 and 30 diploid sequences respectively. Later, the validation and testing diploid sequences were used to generate admixed descendants using Wright-Fisher forward simulation over a series of generations. From 30 diploid sequences of single-ancestry individuals, a total of 100 diploid sequences representing 100 admixed individuals were generated with the admixture event occurring 8 generations in their past to create both validation and testing sets.

The 240 diploid sequences representing 240 single-ancestry individuals were used to train RFMix. The same diploid sequences are used to train generative machine learning model 600 as a CVAE-CGAN model (provided as input sequence x and real sequence xᵣₑₐₗ). Moreover, the 100 diploid sequences representing 100 admixed individuals, generated using Wright-Fisher forward simulation, were used to evaluate RFMix following training. In this experiment, diploid sequences of chromosome 20 are simulated.

From the experiment, 80 simulated samples per ancestry are generated using generative machine learning model 600 and used to train RFMix. RFMix is then evaluated with the 100 diploid sequences of admixed individuals. RFMix is also trained with the 240 diploid sequences representing 240 single-ancestry individuals representing the out-of-Africa dataset and then evaluated again with the same 100 diploid sequences of admixed individuals. The inference accuracies of local-ancestry inference by RFMix trained with the two different datasets are then compared. Table 1 below illustrates the experiment results:

TABLE 1
Accuracy of RFMix trained with out-of-Africa dataset and dataset
generated by generative machine learning model 600 of FIG. 6

| | RFMix Validation | RFMix Test |
|---|---|---|
| Training Method | Accuracy | Accuracy |
| Out-of-Africa dataset | 97.98% | 97.75% |
| Simulated data from CVAE | 93.21% | 93.05% |
| Simulated data from CVAE- | 97.58% | 97.72% |
| CGAN | | |

As shown in Table 1 above, RFMix obtains comparable accuracies when trained with out-of-Africa data and dataset generated by generative machine learning model 600. The accuracy results also show that adding the discriminator and the adversarial loss helps the network to learn to simulate human-chromosome sequences that are more similar to the Out-of-Africa dataset and therefore more useful to train a local-ancestry inference model, such as RFMix, thereby providing a significant increase in accuracy.

### C. Global Dataset

In a second experiment, RFMix and generative machine learning model 600 are trained using SNP sequences of a total of 258 single-population individuals from East Asia (EAS), African (AFR) and European (EUR) ancestry. Specifically, the SNP sequences of 83 Han Chinese in Beijing, China (CHB), 88 Yoruba in Ibadan, Nigeria (YRI) and 87 Iberian Population in Spain (IBS), are used in the second experiment. Additionally, 10 single-individuals per ancestry are used to generate admixed descendants for testing and validation using Wright-Fisher forward simulation over a series of generations. From the SNP sequences of 30 single-ancestry individuals, SNP sequences of a total of 100 admixed individuals are generated with the admixture event occurring 12 generations in their past to create both validation and testing sets. The SNP sequences of the 258 single-ancestry individuals are used to train RFMix and the class-conditional VAE-GAN (CVAE-CGAN), whereas the SNP sequences of the 200 admixed individuals of the validation and testing sets are used to evaluate RFMix following training. In this experiment, chromosome 20 of each individual are used.

The SNP sequences of the 258 single-ancestry individuals are used to train a CVAE-CGAN for each ancestry. After training, a total of 100 simulated SNP sequences are generated per ancestry and used to train RFMix. RFMix is then evaluated with the SNP sequences of the 100 admixed individuals in the validation set. Hyper-parameters of the CVAE-CGAN, including W (the number of SNPs per segment), H (the size of hidden layer), and J (the number of dimensions of latent space), as well as training parameters such as learning rate, batch size and epoch, are selected to provide the highest validation accuracy of RFMix. Specifically W=4000, H=100, and J=10 are selected. In addition, two types of ancestral origin indicators are used—one-hot encoding to select one out of three ancestral origins (C=3), and coordinates of ancestral origin locale (C=2).

From the experiment, the 100 simulated samples per ancestry are generated using generative machine learning model 600 are used to train RFMix. RFMix is then evaluated with the 200 SNP sequences of admixed individuals. RFMix is also trained with the SNP sequences of the 258 single-ancestry individuals and then evaluated again with the same 200 SNP sequences of admixed individuals. The inference accuracies of local-ancestry inference by RFMix trained with the two different datasets are then compared. Table 2 below illustrates the experiment results:

TABLE 2
Accuracy of RFMix trained with single-ancestry dataset and dataset
generated by generative machine learning model 600 of FIG. 6

| | RFMix Val. | RFMix Test |
|---|---|---|
| Method | Accuracy | Accuracy |
| Single-ancestry dataset | 95.57% | 95.33% |
| Generated Data (CVAE) | 91.81% | 91.55% |
| Generated Data (CVAE- | 95.60% | 95.05% |
| CGAN) with one-hot encoded | | |
| ancestral origin indicator | | |
| Generated Data (CVAE- | 95.15% | 95.22% |
| CGAN) with coordinates as | | |
| ancestral origin indicator | | |

As shown in Table 2 above, RFMix obtains comparable accuracies when trained with out-of-Africa data and dataset generated by generative machine learning model 600. The accuracy results also show that adding the discriminator and the adversarial loss helps the network to learn to simulate human-chromosome sequences that are more similar to the Out-of-Africa dataset and therefore more useful to train a local-ancestry inference model, such as RFMix, thereby providing a significant increase in accuracy.

In addition, an analysis of similarity between the simulated SNP sequences (generated by generative machine learning model 600 from the 258 single-ancestry individuals) and the real SNP sequences of the 258 single-ancestry individuals is performed. An extensive sampling of simulated SNP sequences is performed, and the frequency of a simulated individual matching the SNP sequences of one of the 258 single-ancestry individuals with 99.9%, 99.99%, 99.999% and 100% thresholds are determined. Table 3 below shows the number of matches after generating 10,000 SNP sequences representing 10,000 individuals per ancestry:

TABLE 3
Synthetic individuals (out of 10,000) that have P % of
SNPs matching those of a single-ancestry individual

| P | 99.9% | 99.99% | 99.999% | 100% |
|---|---|---|---|---|
| Number of Individuals | 2974 | 266 | 30 | 7 |

### V. Combining Segments

In the example of FIG. 6, encoder 602 and decoder 604 can be trained for a particular window of the genome. Then, an input sequence can be provided along with a trait indicator to generate a simulated sequence that is indistinguishable from a real sequence with the same trait. A separate model can be trained for each genomic window. Thus, each window of a simulated genome can be generated independently. However, it may be desirable for the windows to be interconnected with the simulated sequences for multiple windows (segments) being generated collectively based on an input sequencing spanning the windows.

To provide an interconnection, embodiments can add one or more layers of a model that receive the input vectors and/or embedding vectors for multiple windows. For example, extra layers can exist for a neural network that interconnect the neural networks for different windows. In this manner, the simulated sequence can more realistically simulate a combined genome that is affected by windows being associated with different traits. The long relationships between distant sites can be captured for a given trait. For example, one window can have an ancestral origin of Spanish and another window can have an ancestral original of Native American, and the interconnection can simulate a real-world modern Latino person.

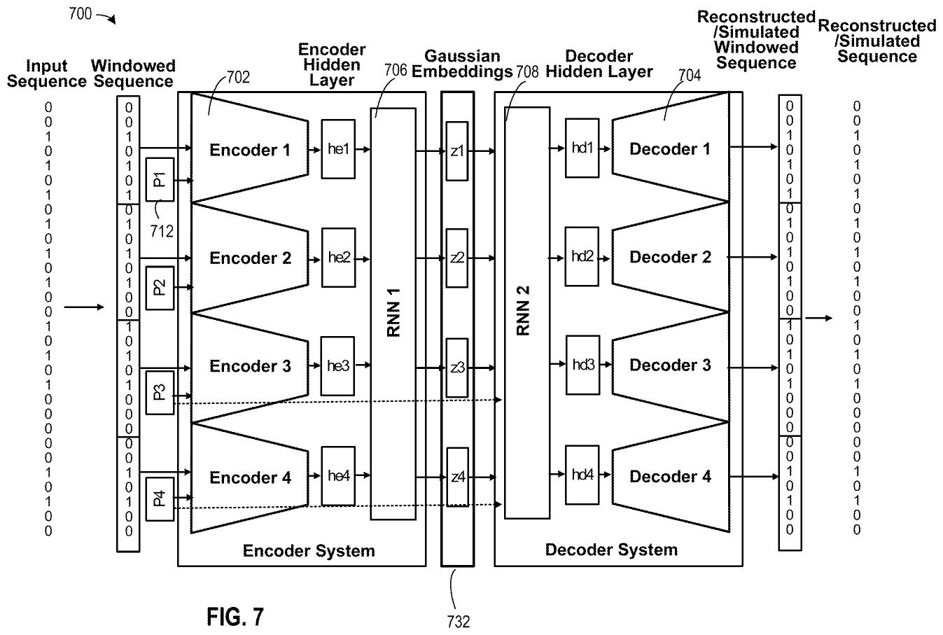

FIG. 7 shows a sample architecture of a machine learning model 700 that provides relationships among different variant segments according to embodiments of the present disclosure. Machine learning model 700 can be used if the input sequence is very long, or modeling of the segments as subsequences is desired (e.g. in order to simulate individuals having mixed traits). The entire input sequence can be viewed as a single segment of windows or multiple segments, with each segment corresponding to a different window. In the latter scenario, the segments can form a larger region or super segment.

The input sequence is the entire sequence of the window(s) for which a simulated sequence is desired. As an example, the variant values of 0 or 1 can be whether a non-wildtype allele exists at the site (e.g., a different allele than the reference sequence). Different sites can be associated with different types of variants. The windowed sequence shows the variant values grouped by different variant segments (windows), each corresponding to a respective set of variant sites.

Each set of variant values for a given variant segment is provided as input to a respective encoder 702. As shown, there are four variant segments corresponding to encoders 1-4. Additionally, each trait indicator vector 712 (P1-P4) provides a respective input to the respective encoder 702. The trait indicator vector 712 can provide an indication of whether one or more traits (e.g., phenotypes, ancestry indicators, . . . ) exist for a given window of the input sequence, e.g., as a result of a subject (e.g., from which the window sequence is obtained) having the one or more traits. These indicators/phenotype/trait descriptors can be provided by doctors, or questionnaires, or other techniques for biobank creation, or obtained through external algorithms (e.g., ancestry indicators could be automatically obtained through local-ancestry inference methods).

Each trait indicator vector 712 (P1, P2, . . . ) is input to each encoder 702 of the encoder system and to a decoding interconnection module 708 (RNN2 module as shown) of the decoder system. Thus, each encoder 702 (1, 2, . . . ) can receive the corresponding windowed sequence and a respective trait vector. The two inputs can be concatenated and then input. Decoding interconnection module 708 can receive sees a sequence of Gaussian embeddings concatenated with the trait indicator vectors as inputs.

Each encoder 702 outputs an encoder hidden layer for each window (e.g., a variant segment). Each portion of the encoder hidden layer (e.g., he1) can correspond to the output of the encoder described in previous sections, e.g., for distribution generation sub-model 306. Thus, each portion of the encoder hidden layer can exist in the latent space

An encoding interconnection module 706 receives the outputs of the encoders as the encoder hidden layer. In the example shown, encoding interconnection module 706 is a recurrent neural network (RNN). The encoding interconnection module 706 operates on all of the values in the latency space for each of the encoders (i.e., each of the windows), and thus operates collectively. Encoding interconnection module 706 provides an output that can be the same or different than the size of the latency space for each of the segments (windows) included in the input sequence.

An embedding vector 732 can be determined in a similar manner as described herein, e.g., in section III. As shown, embedding vector 732 is determined using a Gaussian distribution. The sampling of the distributions can be performed after encoding interconnection module 706 as part of generating embedding vector 732, e.g., as described herein for other sections. A decoding interconnection module 708 receives embedding vector 732 and outputs a decoder hidden layer. Decoding interconnection module 708 operates on all of the values of embedding vector 732, which may be in the latency space, and also receives an input of each trait indicator 712 for the windows, and thus can also operate collectively on values for the different windows. The encoding and decoding hidden layers may have the same or different amount of data (e.g., a same number of dimensions), and embedding vector 732 can be the same or different size than the hidden layers.

Each decoder 704 receives a portion (hd1-hd4) of the decoder hidden layer and outputs the variant values for a respective window in the reconstructed/simulated windowed sequence, which results in a final reconstructed/simulated sequence. Each decoder 704 can correspond to decoders described in previous sections, e.g., sequence generation sub-model 308.

Interconnection modules 706 and 708 can treat each he* and hd* as one entry of a sequence. Therefore, embodiments cans include network layers that can model sequences. Although the interconnection modules are named RNN, they do not need to be recurrent neural networks (RNN). Any neural architecture that can model 1d sequences can be applied, or other differentiable function. Such examples include recurrent Neural Networks (RNNs) such as long short-term memory networks (LSTMs) and gated recurrent units (GRUs), 1d Convolutional Neural Networks (CNNs), including ResNet-style architectures, transformer-based such as networks with self-attention layers and any fast variant of transformers, fully-connected sequence modeling: including networks such as multilayer perceptron network (MLP)-Mixer, and graph MLP (gMLP).

As described above, interconnection modules 706 and 708 are optional. If not included, each subsequence (window) will be processed independently and possible correlations between subsequences will not be captured by the machine learning model 700. If removed, the machine learning model 700 can operate in a similar manner as described in FIGS. 3B-6, e.g., acting independently at every different subsequence.

### VI. Method

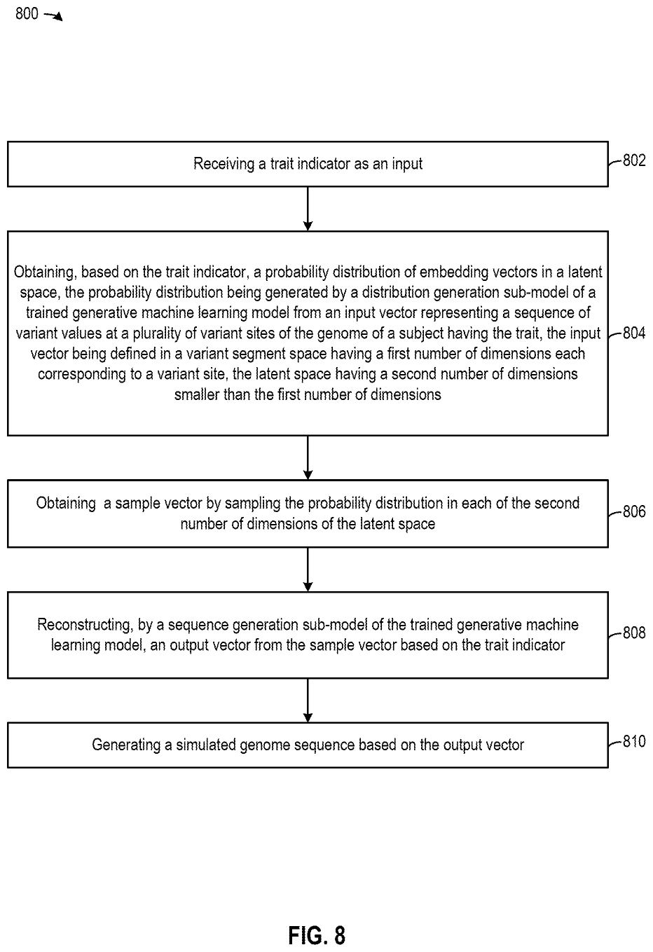

FIG. 8 illustrates a method 800 of generating a simulated genome sequence. The simulated genome sequence may include a sequence of variant (e.g., SNP) values for a plurality of variant (e.g., SNP) sites. Method 800 can be performed by, for example, a computer system that implements a generative machine learning model, such as generative machine learning model 300.

At step 802, the computer system receives a trait indicator as an input. The trait indicator can include, for example, ancestral origin indicator 304 of FIG. 3A or other trait indicator. The computer system may receive other inputs. For example, the computer system may receive an input variant segment (e.g., a SNP segment) for a plurality of variant sites (e.g., SNP sites) of a genome of a subject having a trait associated with the trait indicator.

The variant segment may be represented by a sequence of variant values (e.g., SNP values, other alleles, or a methylation status) at the variant sites. The sequence of variant values can also be referred as an input vector. Each variant value can specify a variant at the variant site. The variant segment can be associated with the trait indicator, e.g., stored with the trait indicator, and associated based on the variant segment being from a subject having the trait. As another example, the computer system may receive information identifying the plurality of variant sites for which the sequence of variant values are generated.

As examples, the trait can be an ancestral origin, a biomedical trait, a demographic trait, or other phenotypes as described herein. Further, more than one trait indicator can be input. In such a situation, the variant segment can be associated with one or more subjects having the plurality of trait indicator that are provided. Accordingly, one or more additional trait indicators corresponding one or more additional traits can be received where the subject also has the one or more additional traits.

In step 804, the computer system obtains, based on the trait indicator, a probability distribution of embedding vectors in a latent space. The probability distribution can be generated, by a distribution generation sub-model of a trained generative machine learning model, from an input vector (e.g., of an input variant segment) representing a sequence of variant values at a plurality of variant sites of the genome of the subject having the trait. For example, the input vector and the trait indicator can be input to the distribution generation sub-model to generate the probability distribution.

Each variant value can specify a particular variant (e.g., a particular base (A, C, G, T), a particular methylation status (methylated or unmethylated), etc.) at a variant site. In some implementations, a 0 can identify a reference value (e.g., allele) in a reference genome or what is other common in a population, and 1 can indicate a presence of a particular type of variant. The input vector can be defined in a variant segment space having a first number of dimensions each corresponding to a variant site. The latent space can have a second number of dimensions smaller than the first number of dimensions. The probability distribution can be considered multi-dimensional with the second number of dimensions.

The type of variant can correspond to class or a characteristics of the variant values at a site. For example, one type of variant is a single nucleotide polymorphisms (SNP), with the variant values being different nucleotides or possibly a deleted nucleotide. Other examples of types of variants are provided herein, such as a deletion, an amplification (e.g., of short tandem repeats), an insertion, an inversion, and a methylation status. The plurality of variant sites have multiple types of variants, e.g., some sites can of SNPs and other sites can be of methylation status.

In some examples, as part of step 804, the computer system can employ a distribution generation sub-model, such as distribution generation sub-model 306, to compute the probability distribution based on an input vector representing an input variant segment. The distribution generation sub-model (e.g., acting as an encoder) can transform the input vector in a variant segment space to a multi-dimensional probability distribution of embedding vectors in a latent space having a reduced number of dimensions, e.g., by mapping to a mean and width (variance) of the distribution for each of the reduced number of dimensions. For an isotropic distribution, the variance would be the same for each dimension. The distributions in the reduced space can represent variations of the input variant segment. The encoder may include a neural network model, which takes, as inputs, the input vector and the ancestral indicator and determines the multi-dimensional probability distribution based on the inputs.

In some examples, the computer system may also select, from a plurality of probability distributions each associated with a particular trait (e.g., ancestral origin) and a set of variant (e.g., SNP) sites, a probability distribution of embedding vectors in the latent space. The probability distributions can be computed by the distribution generation sub-models based on input variant segments for different traits (e.g., ancestral origins) at a prior time. Thus, each of the plurality of probability distributions can be associated with a different trait indicator

In step 806, the computer system obtains a sample vector by sampling the probability distribution in each of the second number of dimensions in the latent space. Specifically, as described with respect to FIG. 3A-FIG. 3E, a random function and a sampling function can be implemented to perform the sampling. The random function can generate a random matrix based on an isotropic Gaussian distribution with a zero mean and a unit-variance. The sampling function can generate a sample vector based on multiplying the output random matrix (from the random function) with a vector of variances of the probability distribution, and adding the result of the multiplication to a vector of means of the probability distribution, in a reparametrization operation.

The probability distribution can comprise a Gaussian distribution (e.g., as described in section III.C), where the probability distribution is represented by a mean and a variance for each dimension of the latent space. Obtaining the sample vector can comprise the following steps for each of the second number of dimensions: generating a random number and combining the random number with the respective mean and the respective variance to generate a value for the dimension. The sample vector can then be formed based on the values generated for the second number of dimensions of the latent space.

In step 808, the computer system reconstructs, using a sequence generation sub-model of the trained generative machine learning model and based on the trait indicator, an output vector from the sample vector. In some examples, sequence generation sub-model can include or be a decoder to implement a reconstruction function. The reconstruction map can map, based on the trait of the input variant segment, samples of embedding vectors in the latent space back to the output vector in the variant segment space. The output vector can then represent a simulated variant segment for a trait. The decoder can also include a neural network model.

Method 800 can be repeated for multiple segments (e.g., as shown in FIGS. 1B, 2A, and 2B). The computer system can receive a plurality of input variant segments extracted from an input genome sequence of one or more subjects having the trait. Each of the plurality of input variant segments can be a separate vector including variant values at variant sites for that segment. The input variant segments can include the input vector, such that the process is repeated for each segment. For each input variant segment, the distribution generation sub-model can determine a probability distribution. A respective sample vector can be obtained by sampling the probability distribution, thereby obtaining a plurality of respective sample vectors. The sequence generation sub-model can reconstruct, based on a respective trait indicator for the segment, a respective output vector from the respective sample vector, thereby determining a plurality of respective output vectors. The simulated genome sequence can then be generated based on (e.g., concatenating) the respective output vectors. The distribution generation sub-model and the sequence generation sub-model can form a class-conditional variational autoencoder (CVAE), where traits of input variant segments can represent different classes for the CVAE.

In step 810, the computer system generates a simulated genome sequence based on the output vector. In some examples, the computer system may receive a plurality of input variant segments and generate a plurality of output vectors representing simulated variant segments. In some examples, the computer system may also generate a plurality of output vectors for different variant sites, with each output vector generated for a particular trait. In both cases, the output vectors can be concatenated to form the simulated genome sequence.

### A. Neural Network Implementation

As described in sections III.C and III.E and other sections herein, the distribution generation sub-model can comprise a first neural network that includes a first input layer, a first hidden layer, and a first output layer. Each node of a first subset of nodes of the first input layer can correspond to a variant site in an input variant segment, can receive a variant value for a corresponding variant site, and can scale the variant value with a first weight of a plurality of first weights. Each node in the first hidden layer can generate a first intermediate value based on a sum of scaled variant values from the first subset of nodes of the first input layer, and can scale the first intermediate value based on a second weight of a plurality of second weights to obtain a scaled first intermediate value. Each node of the first output layer can output the mean and the variance for a dimension of the latent space based on a sum of the scaled first intermediate values from each node of the first hidden layer. The plurality of first weights and the plurality of second weights can be selected based on the trait of the input variant segment.

Each node of a second subset of nodes of the first input layer can receive a value representing the trait of the input variant segment. Each node in the first hidden layer can generate the first intermediate value based on the sum of scaled variant values from the first subset of nodes of the first input layer and a sum of scaled values representing the trait from the second subset of nodes of the first input layer.

As further described in sections III.C and III.E and other sections herein, the sequence generation sub-model can comprise a second neural network that includes a second input layer, a second hidden layer, and a second output layer. Each node of a first subset of nodes of the second input layer can correspond to a dimension of the latent space, can receive a sample vector value for a corresponding dimension, and can scale the sample vector value with a third weight. Each node in the second hidden layer can generate a second intermediate value based on a sum of scaled sample vector values from the first subset of nodes of the second input layer, and can scale the second intermediate value based on a fourth weight. Each node of the second output layer can output a vector value of the respective output vector representing a simulated variant segment. The third weight and the fourth weight can be selected based on the trait of an input variant segment.

Each node of a second subset of nodes of the second input layer can receive a value representing the trait of an input variant segment. Each node in the second hidden layer can generate the second intermediate value based on a sum of scaled variant values from the first subset of nodes of the second input layer and a sum of scaled values representing the trait from the second subset of nodes of the second input layer.

As further described in sections III.C and III.E and other sections herein, the discriminator can comprise a third neural network that includes a third input layer, a third hidden layer, and a third output layer. Each node of a first subset of nodes of the third input layer can correspond to a variant site, can receive a variant value for a corresponding variant site in the output vector, and can scale the variant value with a fifth weight. Each node in the third hidden layer can generate a third intermediate value based on a sum of scaled variant values from the first subset of nodes of the third input layer, and can scale the third intermediate value based on a sixth weight to obtain a scaled third intermediate value. The third output layer can comprise a node to compute, based on the scaled third intermediate values from the third hidden layer, a probability that the output vector represents a real variant segment. The fifth weight and the sixth weight can be selected based on the trait of the input variant segment.

Each node of a second subset of nodes of the third input layer can receive a value representing the trait of the input variant segment. Each node in the third hidden layer can generate the third intermediate value based on the sum of scaled variant values from the first subset of nodes of the third input layer and a sum of scaled values representing the trait from the second subset of nodes of the third input layer.

### B. Training

As described in sections III.E and other sections herein, the encoder (e.g., the distribution generation sub-model) and the decoder (e.g., the sequence generation sub-model) can be part of a CVAE and can be trained to fit different patterns of variants to a target multi-dimensional probability distribution, while reducing the information loss in the mapping from the variant segment space to the latent space. This can ensure that a simulated variant segment generated by the decoder is statistically related to the input variant segment according to the multi-dimensional probability distribution and can simulate the effect of random variations in the variant segment. The training of the encoder and the decoder, as described in FIG. 4, can be based on minimizing a loss function that combines a reconstruction error (between the input vector and each of the output vectors) and a penalty for a divergence from a target probability distribution (e.g., based on differences in the parameters (e.g., mean and variance) of the multi-dimensional probability distribution and target values, e.g., of a target probability distribution). The training operation can performed to reduce or minimize the reconstruction error and the penalty of distribution divergence to force the distribution of variant segments generated by the encoder to match (to a certain degree) the target probability distribution, which can be a zero-mean unit-variance Gaussian distribution. The center (mean) and variance of the distribution of the variant segments can be set based on reducing/minimizing the reconstruction error and the penalty of distribution divergence.

To further reduce the distribution error such that the simulated variant segments can follow the target probability distribution more closely, the CVAE can be trained using a class-conditional generative adversarial network (CGAN), which includes the decoder and a discriminator in the aforementioned training operation, e.g., as described in FIG. 5A and FIG. 5B. The discriminator can also be implemented as a neural network model and can classify whether a variant segment output by the decoder is a real variant segment or a simulated variant segment. The discriminator may be unable to distinguish a real variant segment from a simulated variant segment when the simulated variant segments follow the target probability distribution, at which point the classification error rate of the discriminator may reach a maximum, which means the reconstruction of the decoder is optimal. An adversarial training operation can be performed, in which the parameters of the decoder is adjusted to increase the classification error rate so that the probability distribution in the reduced dimensions approach the target probability distribution, whereas the parameters of the discriminator is adjusted to reduce the classification error rate. The training operation can stop when roughly half of the output vectors represent the real variant segment and roughly half of the output vectors represent fake/simulated variant segment.

As described in sections III.D and III.E and other sections herein, the distribution generation sub-model can be trained based on a first loss function including a reconstruction error component and a distribution error component. The reconstruction error component can be based on a difference between the output vector and the input vector. The distribution error component can be based on a difference between the probability distribution of embedding vectors and a target probability distribution. Parameters of the distribution generation sub-model can be adjusted to decrease the first loss function. The distribution error component can be based on Kullback-Leibler divergence.

The sequence generation sub-model can be trained based on a second loss function including the reconstruction error component. The sequence generation sub-model can be trained in an adversarial training operation with a discriminator that classifies, based on the trait of an input variant segment, whether the output vectors output by the sequence generation sub-model represent real variant sequences or simulated variant sequences. The second loss function can further comprises an adversarial loss component that reduces when a rate of classification error at the discriminator increases. The discriminator can be trained based on a third loss function that decreases when the rate of classification error decreases. Parameters of the sequence generation sub-model and of the discriminator can be adjusted to decrease, respectively, the second loss function and the third loss function.

### C. Collective Analysis of Windows of a Sequence

As described in section V, the plurality of respective output vectors can be reconstructed collectively from the plurality of respective sample vectors. For example, the probability distribution can be determined collectively for the plurality of input variant segments.

For each of the plurality of input variant segments, a respective encoder of the sequence generation sub-model can receive the variant values of the input variant segment and one or more respective trait indicators. Using the one or more respective trait indicators, the respective encoder can operate on the variant values of the input variant segment and output a respective encoder hidden vector (e.g., in a space of size between the variant segment space and the latent space). A plurality of encoder hidden vectors can be obtained. An encoding interconnection module can then receive the plurality of encoder hidden vectors. The encoding interconnection module can generate an embedding vector, which can define the probability distribution for each of the second number of dimensions in the latent space for each of the plurality of input variant segments.

Reconstructing the plurality of respective output vectors can be performed collectively using the embedding vector. A decoding interconnection module can receive the embedding vector and the one or more respective trait indicators. Using trait indicators for the plurality of input variant segments, the decoding interconnection module can operate on the embedding vector and output a respective decoder hidden vector for each of the plurality of input variant segments. For each of the plurality of input variant segments, a respective decoder of the sequence generation sub-model can operate on a respective decoder hidden vector to obtain the respective output vector for the input variant segment.

### VII. Computer System

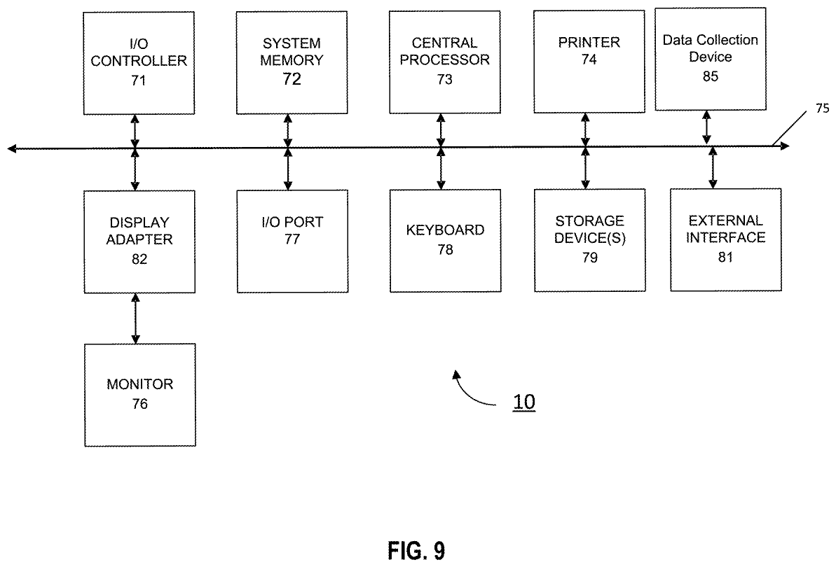

Any of the computer systems mentioned herein may utilize any suitable number of subsystems. Examples of such subsystems are shown in FIG. 9 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices. In some embodiments, a cloud infrastructure (e.g., Amazon Web Services), a graphical processing unit (GPU), etc., can be used to implement the disclosed techniques.

The subsystems shown in FIG. 9 are interconnected via a system bus 75. Additional subsystems such as a printer 74, keyboard 78, storage device(s) 79, monitor 76, which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire). For example, I/O port 77 or external interface 81 (e.g. Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81 or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware (e.g. an application specific integrated circuit or field programmable gate array) and/or using computer software with a generally programmable processor in a modular or integrated manner. As used herein, a processor includes a single-core processor, multi-core processor on a same integrated chip, or multiple processing units on a single circuit board or networked. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective steps or a respective group of steps. Although presented as numbered steps, steps of methods herein can be performed at a same time or in a different order. Additionally, portions of these steps may be used with portions of other steps from other methods. Also, all or portions of a step may be optional. Additionally, any of the steps of any of the methods can be performed with modules, units, circuits, or other means for performing these steps.

The specific details of particular embodiments may be combined in any suitable manner without departing from the spirit and scope of embodiments of the disclosure. However, other embodiments of the disclosure may be directed to specific embodiments relating to each individual aspect, or specific combinations of these individual aspects.

The above description of example embodiments of the disclosure has been presented for the purposes of illustration and description. It is not intended to be exhaustive or to limit the disclosure to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover reference to a “first” or a “second” component does not limit the referenced component to a particular location unless expressly stated.

All patents, patent applications, publications, and descriptions mentioned herein are incorporated by reference in their entirety for all purposes. None is admitted to be prior art.

Attached to this description is an Appendix that includes additional information regarding certain embodiments. Other terms used in the Appendix also may not (yet) be terms commonly used in the industry.

1. A computer-implemented method for generating a simulated genome sequence, comprising:
receiving a trait indicator;
obtaining, based on the trait indicator, a probability distribution of embedding vectors in a latent space, the probability distribution being generated by a distribution generation sub-model of a trained generative machine learning model from an input vector representing a sequence of variant values at a plurality of variant sites of a genome of a subject having a trait associated with the trait indicator, each variant value specifying a particular variant that exists at a variant site, the input vector being defined in a variant segment space having a first number of dimensions corresponding to the plurality of variant sites, the latent space having a second number of dimensions smaller than the first number of dimensions, wherein the probability distribution is multi-dimensional with the second number of dimensions;
obtaining a sample vector by sampling the probability distribution in each of the second number of dimensions of the latent space;
reconstructing, by a sequence generation sub-model of the trained generative machine learning model and based on the trait indicator, an output vector from the sample vector, the output vector being defined in the variant segment space; and
generating the simulated genome sequence based on the output vector.

2. The method of claim 1, wherein a type of variant for at least one of the plurality of variant sites is a single nucleotide polymorphisms (SNP).

3. The method of claim 2, wherein the plurality of variant sites have multiple types of variants.

4. The method of claim 1, wherein the trait is an ancestral origin.

5. The method of claim 1, wherein the trait is a biomedical trait or a demographic trait.

6. The method of claim 1, further comprising:
receiving one or more additional trait indicators corresponding to one or more additional traits, wherein the subject also has the one or more additional traits.

7. The method of claim 1, wherein obtaining the probability distribution comprises selecting the probability distribution from a plurality of probability distributions each associated with a different trait indicator.

8. The method of claim 1, wherein obtaining the probability distribution comprises inputting the input vector and the trait indicator to the distribution generation sub-model to generate the probability distribution.

9. The method of claim 8, further comprising:
receiving a plurality of input variant segments extracted from an input genome sequence of the subject, each of the plurality of input variant segments including variant values at variant sites, the plurality of input variant segments including the input vector;
for each of the plurality of input variant segments:
determining, by the distribution generation sub-model, a probability distribution;
obtaining a respective sample vector by sampling the probability distribution, thereby obtaining a plurality of respective sample vectors; and
reconstructing, by the sequence generation sub-model and based on a respective trait indicator, a respective output vector from the respective sample vector, thereby determining a plurality of respective output vectors; and
generating the simulated genome sequence based on the respective output vectors.

10. The method of claim 9, wherein the plurality of respective output vectors are reconstructed collectively from the plurality of respective sample vectors.

11. The method of claim 10, wherein determining the probability distribution is performed collectively for the plurality of input variant segments and includes:
for each of the plurality of input variant segments:
receiving, by a respective encoder of the sequence generation sub-model, the variant values of the input variant segments and one or more respective trait indicators; and
operating, by the respective encoder using the one or more respective trait indicators, on the variant values of the input variant segments and outputting a respective encoder hidden vector, thereby obtaining a plurality of encoder hidden vectors;
receiving, by an encoding interconnection module, the plurality of encoder hidden vectors; and
generating, by the encoding interconnection module, an embedding vector that defines the probability distribution for each of the second number of dimensions in the latent space for each of the plurality of input variant segments.

12. The method of claim 11, wherein reconstructing the plurality of respective output vectors is performed collectively using the embedding vector and includes:
receiving, at a decoding interconnection module, the embedding vector and the one or more respective trait indicators;
operating, by the decoding interconnection module using the one or more respective trait indicators for the plurality of input variant segments, on the embedding vector and outputting a respective decoder hidden vector for each of the plurality of input variant segments; and
for each of the plurality of input variant segments:
operating, by a respective decoder of the sequence generation sub-model, on the respective decoder hidden vector to obtain the respective output vector for the input variant segment.

13. The method of claim 9, wherein the probability distribution comprises a Gaussian distribution, wherein the probability distribution is represented by a mean and a variance for each dimension of the latent space, and wherein obtaining the sample vector comprises:
for each of the second number of dimensions:
generating a random number; and
combining the random number with the respective mean and the respective variance to generate a value for the dimension; and
forming the sample vector based on the values generated for the second number of dimensions of the latent space.

14. The method of claim 13, wherein the distribution generation sub-model comprises a first neural network, the first neural network comprising a first input layer, a first hidden layer, and a first output layer,
wherein each node of a first subset of nodes of the first input layer corresponds to a variant site in an input variant segment, receives a variant value for a corresponding variant site, and scales the variant value with a first weight of a plurality of first weights,
wherein each node in the first hidden layer generates a first intermediate value based on a sum of scaled variant values from the first subset of nodes of the first input layer, and scales the first intermediate value based on a second weight of a plurality of second weights to obtain a scaled first intermediate value, and
wherein each node of the first output layer outputs the mean and the variance for a dimension of the latent space based on a sum of the scaled first intermediate values from each node of the first hidden layer.

15. The method of claim 14, wherein each node of a second subset of nodes of the first input layer receives a value representing the trait of the input variant segment, and
wherein each node in the first hidden layer generates the first intermediate value based on the sum of scaled variant values from the first subset of nodes of the first input layer and a sum of scaled values representing the trait from the second subset of nodes of the first input layer.

16. The method of claim 14, further comprising selecting the plurality of first weights and the plurality of second weights based on the trait of the input variant segment.

17. The method of claim 13, wherein the sequence generation sub-model comprises a second neural network, the second neural network comprising a second input layer, a second hidden layer, and a second output layer,
wherein each node of a first subset of nodes of the second input layer corresponds to a dimension of the latent space, receives a sample vector value for a corresponding dimension, and scales the sample vector value with a third weight,
wherein each node in the second hidden layer generates a second intermediate value based on a sum of scaled sample vector values from the first subset of nodes of the second input layer, and scales the second intermediate value based on a fourth weight, and
wherein each node of the second output layer outputs a vector value of the respective output vector representing a simulated variant segment.

18. The method of claim 17, wherein each node of a second subset of nodes of the second input layer receives a value representing the trait of an input variant segment, and
wherein each node in the second hidden layer generates the second intermediate value based on a sum of scaled variant values from the first subset of nodes of the second input layer and a sum of scaled values representing the trait from the second subset of nodes of the second input layer.

19. The method of claim 17, further comprising selecting the third weight and the fourth weight based on the trait of an input variant segment.

20. The method of claim 8, wherein the distribution generation sub-model and the sequence generation sub-model form a class-conditional variational autoencoder (CVAE), and wherein a plurality of traits of a plurality of input variant segments represent different classes for the CVAE.

21-29. (canceled)

30. A computer product comprising a non-transitory computer readable medium storing a plurality of instructions that, when executed, cause a computer system to perform a method for generating a simulated genome sequence, the method comprising:
receiving a trait indicator;
obtaining, based on the trait indicator, a probability distribution of embedding vectors in a latent space, the probability distribution being generated by a distribution generation sub-model of a trained generative machine learning model from an input vector representing a sequence of variant values at a plurality of variant sites of a genome of a subject having a trait associated with the trait indicator, each variant value specifying a particular variant that exists at a variant site, the input vector being defined in a variant segment space having a first number of dimensions corresponding to the plurality of variant sites, the latent space having a second number of dimensions smaller than the first number of dimensions, wherein the probability distribution is multi-dimensional with the second number of dimensions;
obtaining a sample vector by sampling the probability distribution in each of the second number of dimensions of the latent space;
reconstructing, by a sequence generation sub-model of the trained generative machine learning model and based on the trait indicator, an output vector from the sample vector, the output vector being defined in the variant segment space; and
generating the simulated genome sequence based on the output vector.

31-32. (canceled)

33. A system comprising one or more processors configured to perform;
receiving a trait indicator;
obtaining, based on the trait indicator, a probability distribution of embedding vectors in a latent space, the probability distribution being generated by a distribution generation sub-model of a trained generative machine learning model from an input vector representing a sequence of variant values at a plurality of variant sites of a genome of a subject having a trait associated with the trait indicator, each variant value specifying a particular variant that exists at a variant site, the input vector being defined in a variant segment space having a first number of dimensions corresponding to the plurality of variant sites, the latent space having a second number of dimensions smaller than the first number of dimensions, wherein the probability distribution is multi-dimensional with the second number of dimensions;
obtaining a sample vector by sampling the probability distribution in each of the second number of dimensions of the latent space;
reconstructing, by a sequence generation sub-model of the trained generative machine learning model and based on the trait indicator, an output vector from the sample vector, the output vector being defined in the variant segment space; and
generating a simulated genome sequence based on the output vector.

34. (canceled)

35. The computer product of claim 30, wherein obtaining the probability distribution comprises inputting the input vector and the trait indicator to the distribution generation sub-model to generate the probability distribution, wherein the method further comprises:
receiving a plurality of input variant segments extracted from an input genome sequence of the subject, each of the plurality of input variant segments including variant values at variant sites, the plurality of input variant segments including the input vector;
for each of the plurality of input variant segments:
determining, by the distribution generation sub-model, a probability distribution;
obtaining a respective sample vector by sampling the probability distribution, thereby obtaining a plurality of respective sample vectors; and
reconstructing, by the sequence generation sub-model and based on a respective trait indicator, a respective output vector from the respective sample vector, thereby determining a plurality of respective output vectors; and
generating the simulated genome sequence based on the respective output vectors.

36. The computer product of claim 35, wherein the plurality of respective output vectors are reconstructed collectively from the plurality of respective sample vectors, and wherein determining the probability distribution is performed collectively for the plurality of input variant segments and includes:
for each of the plurality of input variant segments:
receiving, by a respective encoder of the sequence generation sub-model, the variant values of the input variant segments and one or more respective trait indicators; and
operating, by the respective encoder using the one or more respective trait indicators, on the variant values of the input variant segments and outputting a respective encoder hidden vector, thereby obtaining a plurality of encoder hidden vectors;
receiving, by an encoding interconnection module, the plurality of encoder hidden vectors; and
generating, by the encoding interconnection module, an embedding vector that defines the probability distribution for each of the second number of dimensions in the latent space for each of the plurality of input variant segments.

37. The computer product of claim 35, wherein the plurality of respective output vectors are reconstructed collectively from the plurality of respective sample vectors, and wherein the probability distribution comprises a Gaussian distribution, wherein the probability distribution is represented by a mean and a variance for each dimension of the latent space, and wherein obtaining the sample vector comprises:
for each of the second number of dimensions:
generating a random number; and
combining the random number with the respective mean and the respective variance to generate a value for the dimension; and
forming the sample vector based on the values generated for the second number of dimensions of the latent space.
